SEQUENCE LISTING (69 sequences)

SEQ ID NO: 1 - protein, 1368 aa, Streptococcus pyogenes [sequence omitted]

SEQ ID NO: 2 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 4 aa, Escherichia coli
CGPC

SEQ ID NO: 4 - protein, 10 aa, Artificial
  Cyclic peptide
TWKTSRISIF

SEQ ID NO: 5 - protein, 10 aa, Artificial
  Cyclic peptide
FGRLVSSIRY

SEQ ID NO: 6 - protein, 12 aa, Artificial
  Cyclic peptide
EPIHRSTLTALL

SEQ ID NO: 7 - protein, 6 aa, Artificial
  Cyclic peptide
HWRGWV

SEQ ID NO: 8 - protein, 6 aa, Artificial
  Cyclic peptide
HYFKFD

SEQ ID NO: 9 - protein, 6 aa, Artificial
  Cyclic peptide
HFRRHL

SEQ ID NO: 10 - protein, 8 aa, Artificial
  Cyclic peptide
NKFRGKYK

SEQ ID NO: 11 - protein, 8 aa, Artificial
  Cyclic peptide
NARKFYKG

SEQ ID NO: 12 - protein, 7 aa, Artificial
  Cyclic peptide
KHRFNKD

SEQ ID NO: 13 - protein, 4 aa, Artificial
  Synthetic peptide linker
GGGG

SEQ ID NO: 14 - protein, 5 aa, Artificial
  Synthetic peptide linker
PSGSP

SEQ ID NO: 15 - protein, 5 aa, Artificial
  Synthetic peptide linker
PSPSP

SEQ ID NO: 16 - protein, 5 aa, Artificial
  Synthetic peptide linker
PSPSP

SEQ ID NO: 17 - protein, 4 aa, Artificial
  Synthetic peptide linker
KKKK

SEQ ID NO: 18 - protein, 4 aa, Artificial
  Synthetic peptide linker
RRRR

SEQ ID NO: 19 - protein, 5 aa, Artificial
  Synthetic peptide linker
ASASA

SEQ ID NO: 20 - protein, 6 aa, Artificial
  Synthetic peptide linker
GGSGGS

SEQ ID NO: 21 - protein, 5 aa, Artificial
  Synthetic peptide linker
GGGGS

SEQ ID NO: 22 - protein, 10 aa, Artificial
  Synthetic peptide linker
GGGGSGGGGS

SEQ ID NO: 23 - protein, 15 aa, Artificial
  Synthetic peptide linker
GGGGSGGGGSGGGGS

SEQ ID NO: 24 - protein, 20 aa, Artificial
  Synthetic peptide linker
GGGGSGGGGSGGGGSGGGGS

SEQ ID NO: 25 - protein, 25 aa, Artificial
  Synthetic peptide linker
GGGGSGGGGSGGGGSGGGGSGGGGS

SEQ ID NO: 26 - protein, 30 aa, Artificial
  Synthetic peptide linker
GGGGSGGGGSGGGGSGGGGSGGGGSGGGGS

SEQ ID NO: 27 - protein, 32 aa, Artificial
  Synthetic peptide linker
GGGGSGGGGSGGGGSFKGGGGSGGGGSGGGGS

SEQ ID NO: 28 - protein, 32 aa, Artificial
  Synthetic peptide linker
GGGGSGGGGSGGGGSVAGGGGSGGGGSGGGGS

SEQ ID NO: 29 - protein, 7 aa, Simian vacuolating virus 40
PKKKRKV

SEQ ID NO: 30 - protein, 16 aa, Xenopus sp
KRPAATKKAGQAKKKK

SEQ ID NO: 31 - protein, 4 aa, Artificial
  Nuclear localization signal consensus sequence
  misc_feature (1)..(): Xaa is any amino acid
  misc_feature (2)..(2): Xaa is K or R
  misc_feature (3)..(3): Xaa is any amino acid
  misc_feature (4)..(4): Xaa is K or R
KXXX

SEQ ID NO: 32 - protein, 7 aa, Simian vacuolating virus 40
PKKKRKV

SEQ ID NO: 33 - protein, 5 aa, Simian vacuolating virus 40
KKKRK

SEQ ID NO: 34 - protein, 5 aa, Adenovirus sp
KRPRP

SEQ ID NO: 35 - protein, 9 aa, Homo Sapiens
PAAKRVKLD

SEQ ID NO: 36 - protein, 11 aa, Homo sapiens
RQRRNELKRSP

SEQ ID NO: 37 - protein, 16 aa, Xenopus sp
KRPAATKKAGQAKKKK

SEQ ID NO: 38 - protein, 24 aa, Xenopus sp
VRKKRKTEEESPLKDKDAKKSKQE

SEQ ID NO: 39 - protein, 24 aa, Mus musculus
RLRRDAGGRGGVYEHLGGAPRRRK

SEQ ID NO: 40 - protein, 19 aa, Homo sapiens
KRKGDEVDGVDECAKKSKK

SEQ ID NO: 41 - protein, 38 aa, Artificial
  Synthetic nuclear targeting peptide
NQSSNFGPMKGGNFGGRSSGPYGGGGQYFAKPRNQGGY

SEQ ID NO: 42 - protein, 5 aa, S. thermophilius
NGGNG

SEQ ID NO: 43 - protein, 6 aa, S. thermophilius
NNAAAW

SEQ ID NO: 44 - protein, 6 aa, S. thermophilius
NNAGAA

SEQ ID NO: 45 - protein, 7 aa, S. thermophilius
NNNGATT

SEQ ID NO: 46 - protein, 7 aa, C. jejuni
NNNNACA

SEQ ID NO: 47 - protein, 8 aa, N. meningitides
NNNNGATT

SEQ ID NO: 48 - protein, 8 aa, P. multocida
GNNNCNNA

SEQ ID NO: 49 - RNA, 21 nt, Artificial
  Guide RNA
  misc_feature (1)..(20): n is a, c, g, or u
nnnnnnnnnnnnnnnnnnnng

SEQ ID NO: 50 - RNA, 23 nt, Artificial
  Guide RNA
  misc_feature (2)..(22): n is a, c, g, or u
gnnnnnnnnnnnnnnnnnnnnng

SEQ ID NO: 51 - RNA, 19 nt, Artificial
  Guide RNA
aauuucuacuguuguagau

SEQ ID NO: 52 - RNA, 19 nt, Artificial
  Guide RNA
aauuucugcuguugcagau

SEQ ID NO: 53 - RNA, 19 nt, Artificial
  Guide RNA
aauuuccacuguuguggau

SEQ ID NO: 54 - RNA, 19 nt, Artificial
  Guide RNA
aauuccuacuguuguaggu

SEQ ID NO: 55 - RNA, 19 nt, Artificial
  Guide RNA
aauuucuacuauuguagau

SEQ ID NO: 56 - RNA, 19 nt, Artificial
  Guide RNA
aauuucuacugcuguagau

SEQ ID NO: 57 - RNA, 18 nt, Artificial
  Guide RNA
aauuucuacuuuguagau

SEQ ID NO: 58 - RNA, 17 nt, Artificial
  Guide RNA
aauuucuacuuguagau

SEQ ID NO: 59 - RNA, 67 nt, Artificial
  Guide RNA
gaauuuuucaacgggugugccaauggccacuuuccagguggcaaagcccguugagcuucucaaaaag

SEQ ID NO: 60 - RNA, 79 nt, Artificial
  Guide RNA
gucuagaggacagaauuuuucaacgggugugccaauggccacuuuccagguggcaaagcccguugagcuucucaaaaag

SEQ ID NO: 61 - RNA, 78 nt, Artificial
  Guide RNA
ucuagaggacagaauuuuucaacgggugugccaauggccacuuuccagguggcaaagcccguugagcuucucaaaaag

SEQ ID NO: 62 - RNA, 36 nt, Artificial
  Guide RNA
acuuuccaggcaaagcccguugagcuucucaaaaag

SEQ ID NO: 63 - RNA, 9 nt, Artificial
  Guide RNA
agcuucuca

SEQ ID NO: 64 - RNA, 8 nt, Artificial
  Guide RNA
gcuucuca

SEQ ID NO: 65 - RNA, 34 nt, Artificial
  Guide RNA
  misc_feature (15)..(34): n is a, c, g, or u
cugagaaguggcacnnnnnnnnnnnnnnnnnnnn

SEQ ID NO: 66 - RNA, 14 nt, Artificial
  Guide RNA
cugagaaguggcac

SEQ ID NO: 67 - RNA, 9 nt, Artificial
  Guide RNA
cugagaagu

SEQ ID NO: 68 - RNA, 13 nt, Artificial
  Guide RNA
ugagaaguggcac

SEQ ID NO: 69 - RNA, 8 nt, Artificial
  Guide RNA
ugagaagu

(19) United States
(12) Patent Application Publication
(10) Pub. No.: US 2020/0362038 A1
(43) Pub. Date: Nov. 19, 2020

(54) ANTIBODY-MEDIATED DELIVERY OF CAS9 TO MAMMALIAN CELLS

(71) Applicant: The Regents of the University of California, Oakland, CA (US)

(72) Inventors: Jacob Corn, Zurich (CH); Mark Dewitt, El Cerrito, CA (US); Arik Shams, Berkeley, CA (US); Dana V. Foss, Berkeley, CA (US)

(21) Appl. No.: 16/645,785

(22) PCT Filed: Sep. 10, 2018
(86) PCT No.: PCT/US18/50299

§ 371 Date: Mar. 9, 2020

Related U.S. Application Data
(60) Provisional application No. 62/557,021, filed on Sep. 11, 2017.

(51) Int. Cl. C07K 16/28; C12N 9/22; C12N 15/11; C12N 15/90

(52) U.S. Cl. CPC C07K 16/2809; C12N 9/22; C12N 15/11; C12N 2320/32; C12N 2310/20; C12N 2800/80; C07K 2319/33; C12N 15/907

(57) ABSTRACT

In certain embodiments a construct for performing gene editing in a mammalian cell is provided. In certain embodiments the construct comprises a targeting moiety that binds a surface marker on a cell (e.g., surface receptor), where the targeting moiety is attached to a complex comprising a class 2 CRISPR/Cas endonuclease complexed with a corresponding CRISPR/Cas guide RNA that hybridizes to a target sequence within the genomic DNA of the cell.

### CROSS-REFERENCE TO RELATED APPLICATIONS

This application claims benefit of and priority to U.S. Ser. No. 62/557,021, filed on Sep. 11, 2017, which is incorporated herein by reference in its entirety for all purposes.

### STATEMENT OF GOVERNMENTAL SUPPORT

[Not Applicable]

### BACKGROUND

Considerable attention has been devoted to developing reagents and methods for delivering bioactive agents to particular tissues, cells, and/or subcellular locations. For example, delivery of large molecules, such as an antisense or RNAi molecules or proteins, is difficult as such compounds are generally not able to penetrate cell membranes; when they can, they can often lack of selectivity for the tissue of interest and therefore increase risk of off-target pharmacology and present serious safety concerns. Furthermore, selective drug delivery to targeted delivery sites is often a challenge because molecules that are cell permeable are often not selective.

In the context of gene therapy, most gene editing agents have often been delivered via plasmid DNA encapsulated in viral-derived vectors such as adeno viruses and adeno-associated viruses. Unfortunately, this type of approach has been plagued with serious issues for the patients mainly i) increased risk of insertional mutagenesis, ii) increased risks of hepatotoxicity upon interaction of viral vector with Kupffer cells and iii) only transient pharmacological benefit for the patient triggered by immunogenic response against the treated cells. Attempts to find more effective and safer ways to deliver gene editing agents thus far have been elusive.

### SUMMARY

In various embodiments methods and compositions are provided that delivery of Cas effector RNPs to cells without electroporation. In particular, it was a surprising discovery that antibody-mediated uptake of Cas effectors complexed with guide RNA can specifically edit cells expressing high levels of a receptor (target) for the antibody over cells with low levels of the antibody receptor. While targeting of a Cas effector to cells is demonstrated using antibodies as targeting moieties, it is believed that other targeting moieties can also be used (see, e.g., FIG. 5). Such targeting moieties include, inter alia, a DNA aptamer, an RNA aptamer, a peptide aptamer, an anticalin, a lectin, a DARPIN, an antibody, and the like.

Various embodiments contemplated herein may include, but need not be limited to, one or more of the following:

### Embodiment 1

A construct for performing gene editing in a mammalian cell, said construct comprising:
a targeting moiety that binds a cell surface marker, where said targeting moiety is attached to a ribonucleoprotein complex comprising a class 2 CRISPR/Cas endonuclease complexed with a corresponding CRISPR/Cas guide RNA that hybridizes to a target sequence within the genomic DNA of the cell.

### Embodiment 2

The construct of embodiment 1, wherein said targeting moiety is selected from the group consisting of an antibody, a DNA/RNA or peptide aptamer, an anticalin, a lectin, and a DARPIN.

### Embodiment 3

The construct according to any one of embodiments 1-2, wherein said class 2 CRISPR/Cas endonuclease is a type II CRISPR/Cas endonuclease.

### Embodiment 4

The construct according to any one of embodiments 1-3, wherein the class 2 CRISPR/Cas endonuclease is a Cas9 polypeptide and the corresponding CRISPR/Cas guide RNA is a Cas9 guide RNA.

### Embodiment 5

The construct of embodiment 4, wherein said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof.

### Embodiment 6

The construct of embodiment 5, wherein said Cas9 protein comprises a Streptococcus pyogenes Cas9 protein (spCas9).

### Embodiment 7

The construct of embodiment 5, wherein said Cas9 protein comprises a Staphylococcus aureus Cas9 protein (saCas9).

### Embodiment 8

The construct of embodiment 5, wherein said Cas9 protein comprises a Streptococcus thermophilus Cas9 protein.

### Embodiment 9

The construct of embodiment 5, wherein said Cas9 protein comprises a Neisseria meningitides Cas9 protein (nmCas9).

### Embodiment 10

The construct of embodiment 5, wherein said Cas9 protein comprises a Treponema denticola Cas9 protein (tdCas9).

### Embodiment 11

The construct according to any one of embodiments 1-3, wherein the class 2 CRISPR/Cas endonuclease is a the class 2 CRISPR/Cas endonuclease is a high fidelity (HiFi) mutant Cas9 polypeptide and the corresponding CRISPR/Cas guide RNA is a Cas9 guide RNA.

### Embodiment 12

The construct of embodiment 11, wherein said mutant cas9 comprises an Alt-R® CRISPR-Cas9.

### Embodiment 13

The construct of embodiment 11, wherein said mutant cas9 comprises an.R691A Cas9 mutant.

### Embodiment 14

The construct according to any one of embodiments 11-13, wherein said mutant cas9 comprises a Cas9 enhanced with one, two, or three nuclear localization signals (NLS).

### Embodiment 15

The construct of embodiment 14, wherein said NLS comprise an NLS selected from the group consisting of the SV40 T antigen (PKKKRKV (SEQ ID NO:32)), the SV40 Vp3 (KKKRK (SEQ ID NO:33)), the Adenovirus Ela (KRPRP (SEQ ID NO:34)), the human c-myc (PAAKRVKLD (SEQ ID NO:35), RQRRNELKRSP (SEQ ID NO:36)), nucleoplasmin (KRPAATKKAGQAKKKK (SEQ ID NO:37)), Xenopus N1 (VRKKRKTEEESPLKDKDAKKSKQE (SEQ ID NO:38)), mouse FGF3 (RLRRDAGGRGGVYEHLGGAPRRRK (SEQ ID NO:39)); PARP (KRKGDEVDGVDECAKKSKK (SEQ ID NO:40)), M9 peptide, NQSSNFGPMKGGNFGGRSSGPYGGGGQYFAKPRNQGGY (SEQ ID NO:41), and derivatives thereof.

### Embodiment 16

The construct according to any one of embodiments 1-3, wherein the class 2 CRISPR/Cas endonuclease is a type V or type VI CRISPR/Cas endonuclease.

### Embodiment 17

The construct of embodiment 16, wherein the class 2 CRISPR/Cas polypeptide is selected from the group consisting of a Cpf1 polypeptide or a functional portion thereof, a C2c1 polypeptide or a functional portion thereof, a C2c3 polypeptide or a functional portion thereof, and a C2c2 polypeptide or a functional portion thereof.

### Embodiment 18

The construct of embodiment 17, wherein the class 2 CRISPR/Cas polypeptide comprises a Cpf1 polypeptide.

### Embodiment 19

The construct according to any one of embodiments 1-18, wherein said guide RNA comprises one or more bridged nucleic acids.

### Embodiment 20

The construct of embodiment 19, wherein said bridged nucleic acid comprises one or more N-methyl substituted BNAs (2′,4′-BNA^{NC}[N-Me]).

### Embodiment 21

The construct of embodiment 19, wherein said guide RNA comprises one or more locked nucleic acids (LNAs).

### Embodiment 22

The construct according to any one of embodiments 1-21, wherein said targeting moiety binds to an internalizing receptor.

### Embodiment 23

The construct according to any one of embodiments 1-22, wherein said targeting moiety binds to a receptor selected from the group consisting of CD45, CD3, erbB2, Her2, CD22, CD74, CD19, CD20, CD33, CD40, MUC1, IL-15R, HLA-DR, EGP-1, EGP-2, G250, prostate specific membrane antigen (PSMA), prostate specific antigen (PSA), prostatic acid phosphatase (PAP), and placental alkaline phosphatase.

### Embodiment 24

The construct of embodiment 23, wherein said targeting moiety binds to CD45.

### Embodiment 25

The construct of embodiment 23, wherein said targeting moiety binds to CD33.

### Embodiment 26

The construct of embodiment 23, wherein said targeting moiety binds to CD3.

### Embodiment 27

The construct according to any one of embodiments 1-23, wherein said targeting moiety comprises an antibody.

### Embodiment 28

The construct of embodiment 27, wherein said targeting moiety comprises an internalizing antibody.

### Embodiment 29

The construct of embodiment 27, wherein said targeting moiety comprises an anti-CD3 antibody.

### Embodiment 30

The construct of embodiment 29, wherein said antibody comprises an antibody selected form the group consisting of OKT3, m291, foralumab, and CA-3.

### Embodiment 31

The construct of embodiment 29, wherein said antibody comprises OKT3.

### Embodiment 32

The construct of embodiment 27, wherein said targeting moiety comprises an anti-CD45 antibody.

### Embodiment 33

The construct of embodiment 27, wherein said targeting moiety comprises an anti-CD33 antibody.

### Embodiment 34

The construct according to any one of embodiments 27-33, wherein said antibody is a full-length immunoglobulin.

### Embodiment 35

The construct according to any one of embodiments 27-33, wherein said antibody is selected from the group consisting of Fv, Fab, (Fab′)₂, (Fab′)₃, IgGΔCH2, a unibody, and a minibody.

### Embodiment 36

The construct according to any one of embodiments 27-33, wherein said antibody is, wherein said antibody is a single chain antibody.

### Embodiment 37

The construct of embodiment 36 wherein said antibody is an scFv.

### Embodiment 38

The construct according to any one of embodiments 27-37, wherein said antibody is a human antibody.

### Embodiment 39

The construct according to any one of embodiments 1-38, wherein said targeting moiety is attached to said Cas endonuclease by a non-covalent interaction.

### Embodiment 40

The construct of embodiment 39, wherein said non-covalent interaction comprises a biotin/avidin interaction.

### Embodiment 41

The construct of embodiment 39, wherein said non-covalent interaction comprises an interaction between an antibody-binding peptide and said targeting moiety.

### Embodiment 42

The construct according to any one of embodiments 39 or 41, wherein said non-covalent interaction comprises an interaction between said targeting moiety and an antibody binding protein selected from the group consisting of Protein A, Protein G, Protein L, Protein Z, Protein LG, Protein LA, and Protein AG.

### Embodiment 43

The construct according to any one of embodiments 39 or 41, wherein said non-covalent interaction comprises an interaction between said targeting moiety and a binding moiety selected from the group consisting of PAM, D-PAM, D-PAM-θ, TWKTSRISIF (SEQ ID NO:4), FGRLVSSIRY (SEQ ID NO:5, Fc-III, EPIHRSTLTALL, HWRGWV (SEQ ID NO:7), HYFKFD (SEQ ID NO:8), HFRRHL (SEQ ID NO:9), NKFRGKYK (SEQ ID NO:10), NARKFYKG (SEQ ID NO:11), KHRFNKD (SEQ ID NO:12).

### Embodiment 44

The construct according to any one of embodiments 39 or 41, wherein said non-covalent interaction comprises an interaction between said targeting moiety and FcB6.1 peptide.

### Embodiment 45

The construct according to any one of embodiments 41-44, wherein said antibody-binding peptide or said binding moiety is chemically conjugated to said Cas endonuclease via a cleavable linker.

### Embodiment 46

The construct of embodiment 45, wherein said linker comprises a cleavable linker.

### Embodiment 47

The construct of embodiment 53, wherein said cleavable linker comprises a disulfide linker or an acid-labile linker.

### Embodiment 48

The construct of embodiment 47, wherein said linker comprises an acid label linker comprising a moiety selected from the group consisting of a hydrazone, an acetal, a cis-aconitate-like amide, a silyl ether.

### Embodiment 49

The construct of embodiment 47, wherein said linker comprises a Phe-Lys, or a Val-Cit.

### Embodiment 50

The construct according to any one of embodiments 1-38, wherein said targeting moiety is chemically conjugated to said Cas endonuclease.

### Embodiment 51

The construct of embodiment 50, wherein said targeting moiety is chemically conjugated to said Cas endonuclease via a non-cleavable linker.

### Embodiment 52

The construct of embodiment 50, wherein said targeting moiety is chemically conjugated to said Cas endonuclease via a cleavable linker.

### Embodiment 53

The construct of embodiment 50, wherein said targeting moiety is chemically conjugated to said Cas endonuclease via a cleavable linker comprising a disulfide linker or an acid-labile linker.

### Embodiment 54

The construct of embodiment 50, wherein said targeting moiety is chemically conjugated to said Cas endonuclease via an acid label linker comprising a moiety selected from the group consisting of a hydrazone, an acetal, a cis-aconitate-like amide, a silyl ether.

### Embodiment 55

The construct of embodiment 50, wherein said targeting moiety is chemically conjugated to said Cas endonuclease via a non-amino acid, non-peptide linker shown in Table 2.

### Embodiment 56

The construct according to any one of embodiments 1-38, wherein said targeting moiety comprises a polypeptide and said targeting moiety and Cas endonuclease comprise a fusion protein.

### Embodiment 57

The construct of embodiment 56, wherein said fusion protein comprises said targeting moiety directly attached to said Cas endonuclease.

### Embodiment 58

The construct of embodiment 56, wherein said fusion protein comprises said targeting moiety attached to said Cas endonuclease by an amino acid.

### Embodiment 59

The construct of embodiment 56, wherein said fusion protein comprises said targeting moiety attached to said Cas endonuclease by a peptide linker.

### Embodiment 60

The construct of embodiment 59, wherein said linker comprises an amino acid sequence cleavable by a protease.

### Embodiment 61

The construct of embodiment 60, wherein said linker comprises an amino acid sequence cleavable by a cathepsin.

### Embodiment 62

The construct according to any one of embodiments 59-61, wherein said peptide linker comprises a dipeptide valine-citrulline (Val-Cit), or Phe-Lys.

### Embodiment 63

The construct of embodiment 56, wherein said fusion protein comprises said targeting moiety attached to said Cas endonuclease by an amino acid or peptide linker shown in Table 2.

### Embodiment 64

A pharmaceutical formulation, said formulation comprising a construct according to any one of embodiments 1-63, and a pharmaceutically acceptable carrier.

### Embodiment 65

The formulation of embodiment 64, wherein said formulation is for administration via a modality selected from the group consisting of intraperitoneal administration, topical administration, oral administration, inhalation administration, transdermal administration, subdermal depot administration, and rectal administration.

### Embodiment 66

The formulation according to any one of embodiments 64-65, wherein said formulation is a unit dosage formulation.

### Embodiment 67

A method of performing gene editing on a cell, said method comprising contacting said cell with a construct according to any one of embodiments 1-63, wherein said guide RNA guides the Cas endonuclease to a specific location in the genome of said cell.

### Embodiment 68

The method of embodiment 67, wherein said cell is a cell ex vivo.

### Embodiment 69

The method of embodiment 68, wherein said cell is a cell derived from a subject that is to be treated.

### Embodiment 70

The method according to any one of embodiments 68-69, wherein said cell comprise a cell selected from the group consisting of fibroblasts, blood cells (e.g., red blood cells, white blood cells), liver cells, kidney cells, neural cells, and stem cells (e.g., embryonic stem cells, adult stem cells (e.g., hematopoietic stem cells, neuronal stem cells and mesenchymal stem cells), T-cells, and induced pluripotent stem cells (iPSCs).

### Embodiment 71

The method of embodiment 67, wherein said cell is in vivo in a subject and said contacting comprises administering said composition to said subject.

### Embodiment 72

The method of embodiment 71, wherein said method comprises administering said construct via a route selected from the group consisting of intraperitoneal administration, topical administration, oral administration, inhalation administration, transdermal administration, subdermal depot administration, and rectal administration.

### Embodiment 73

The method according to any one of embodiments 71-72, wherein said method comprises administering a pharmaceutical formulation according to any one of embodiments 64-66.

### Embodiment 74

The method according to any one of embodiments 69-73, wherein said subject is a human.

### Embodiment 75

The method according to any one of embodiments 69-73, wherein said subject is a non-human mammal.

### Embodiment 76

The method of any one of embodiments 67-75, wherein said method further comprise introducing a donor template nucleic acid into said cell.

### Embodiment 77

The method according to any one of embodiments 67-76. wherein said method comprises treatment of a disease selected from the group consisting of achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, Crigler-Najjer Syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), Glycogen Storage Disease Type IV, hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome. Other such diseases include, e.g., acquired immunodeficiencies, lysosomal storage diseases (e.g., Gaucher's disease, GM1, Fabry disease and Tay-Sachs disease), mucopolysaccahidosis (e.g., Hunter's disease, Hurler's disease), hemoglobinopathies (e.g., sickle cell diseases, HbC, α-thalassemia, β-thalassemia), and hemophilias.

### Embodiment 78

The method according to any one of embodiments 67-76, wherein said method comprises treatment of a cancer.

### Embodiment 79

The method of embodiment 78, wherein:
the cancer comprises a solid tumor; and/or
the cancer comprises cancer stem cells; and/or
the cancer comprises a cancer selected from the group consisting of breast cancer, prostate cancer, colon cancer, cervical cancer, ovarian cancer, pancreatic cancer, renal cell (kidney) cancer, glioblastoma, acute lymphoblastic leukemia (ALL), acute myeloid leukemia (AML), Adrenocortical carcinoma, AIDS-related cancers (e.g., kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, bronchial tumors, burkitt lymphoma, carcinoid tumors (e.g., childhood, gastrointestinal), cardiac tumors, chordoma, chronic lymphocytic leukemia (CLL), chronic myelogenous leukemia (CML), chronic myeloproliferative disorders, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

### Embodiment 80

The method of embodiment 78, wherein said cancer comprises a liquid cancer (e.g., a leukemia).

### Embodiment 81

The method of embodiment 78, wherein said cancer comprises a solid tumor (e.g. a melanoma).

### Embodiment 82

The method according to any one of embodiments 78-81, wherein said cell comprises a T-cell.

### Embodiment 83

The method of embodiment 82, wherein said method reactivates said T cell.

### Embodiment 84

The method according to any one of embodiments 78-81, wherein said cell comprises a stromal cell.

### Definitions

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment which includes a delay in the onset or the prevention of the onset of a pathology or disease.

As used herein, the term “selective targeting” or “specific binding” refers to use of targeting ligands comprising a construct described here rein. In certain embodiments the targeting ligand(s) are attached to a Cas endonuclease complexed to a guide RNA. Typically the ligands interact specifically/selectively with receptors or other biomolecular components expressed on the target, e.g., a cell surface of interest. The targeting ligands can include such molecules and/or materials as peptides, antibodies, aptamers, targeting peptides, polysaccharides, and the like.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers

An “avidin/biotin” interaction refers to binding reaction between biotin and avidin or avidin variants including but not limited to streptavidin, neutravidin, and the like.

A “pharmaceutically acceptable carrier” as used herein includes, but need not be limited to, any of the standard pharmaceutically acceptable carriers. The pharmaceutical compositions contemplated herein can be formulated according to known methods for preparing pharmaceutically useful compositions. The pharmaceutically acceptable carrier can include diluents, adjuvants, and vehicles, as well as carriers, and inert, non-toxic solid or liquid fillers, diluents, or encapsulating material that does not react with the active ingredients of the invention. Examples include, but are not limited to: phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19th ed.) describes formulations which can be used in connection with the drug delivery nanocarrier(s) (e.g., LB-coated nanoparticle(s)) described herein.

As used herein, an “antibody” refers to a protein consisting of one or more polypeptides substantially encoded by immunoglobulin genes or fragments of immunoglobulin genes or derived therefrom that is capable of binding (e.g., specifically binding) to a target (e.g., to a target polypeptide). The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Certain preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on a phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). In certain embodiments antibodies should include all that have been displayed on phage (e.g., scFv, Fv, Fab and disulfide linked Fv (see, e.g, Reiter et al. (1995) Protein Eng. 8: 1323-1331) as well as affibodies, nanobodies, unibodies, and the like.

The term “specifically binds”, as used herein, when referring to a biomolecule (e.g., protein, nucleic acid, antibody, etc.), refers to a binding reaction that is determinative of the presence of a biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

In class 2 CRISPR systems, the functions of the effector complex (e.g., the cleavage of target DNA) are carried out by a single endonuclease (e.g., see Zetsche et al. (2015) Cell, 163(3):759-771; Makarova et al. (2015) Nat. Rev. Microbiol. 13(11): 722-736; and Shmakov et al. (2015) Mol. Cell. 60(3): 385-397). As such, the term “class 2 CRISPR/Cas protein” is used herein to encompass the endonuclease (the target nucleic acid cleaving protein) from class 2 CRISPR systems. Thus, the term “class 2 CRISPR/Cas endonuclease” as used herein encompasses type II CRISPR/Cas proteins (e.g., Cas9), type V CRISPR/Cas proteins (e.g., Cpf1, C2c1, C2C3), and type VI CRISPR/Cas proteins (e.g., C2c2). To date, class 2 CRISPR/Cas proteins encompass type II, type V, and type VI CRISPR/Cas proteins, but the term is also meant to encompass any class 2 CRISPR/Cas protein suitable for binding to a corresponding guide RNA and forming an RNP complex.

### BRIEF DESCRIPTION OF THE DRAWINGS

FIG. 1 shows the amino acid sequence (SEQ ID NO:1) of an Streptococcus pyogenes Cas9.

FIG. 2 shows the amino acid sequence (SEQ ID NO:2) of an Francisella tularensis cpf1.

FIG. 3 schematically illustrates antibodies conjugated to Cas9 via a biotin/streptavidin linkage.

FIG. 4 illustrates the use of a chemically conjugated antibody-binding protein to attach an antibody to a CRISPR/Cas endonuclease ribonucleoprotein complex.

FIG. 5 schematically illustrates a cell targeting moiety (e.g., a DNA aptamer, an RNA aptamer, a peptide aptamer, an anticalin, a lectin, a DARPIN, an antibody, etc.) attached to Cas effector (e.g., a complex comprising a class 2 CRISPR/Cas endonuclease and a guide RNA) by a linker (e.g., a cleavable or non-cleavable linker).

FIG. 6 shows that Cas9-Ab conjugates are stable. Multiple conjugates=multiple Cas9/Ab through tetrameric streptavidin. Not optimized for 1:1 stoichiometry

FIG. 7 shows that assay editing by silencing BFP correlates very well with ddPCR and NGS.

FIG. 8 shows that Cas9-anti-CD45 selectively edits CD45 high cells.

FIG. 9, panels A and B, illustrates the structures of LNA (2′,4′-BNA) (FIG. 9, panel A), and BNA^{NC} (2′,4′-BNA^{NC} [NMe]) (FIG. 9, panel B).

FIG. 10, panels A-C, illustrates engineering Cas9 for cell targeting and endosomal escape. Panel A: An Ab-Cas9 RNP complex for T cell-specific genome editing. The Cas9 protein (cyan) is fused to a protein A fragment (yellow), which tightly binds a the OKT3 antibody (blue) that induces T cell-specific binding and internalization. The RNP contains a guide RNA (gray). Panel B Nucleofection of the Cas9prA fusion (left) produces similar levels of CD4 KO to that of unmodified Cas9 RNP (right). Cells observed at 3 days; technical duplicates. Panel C: A pronounced shift in retention volume demonstrates complex formation between Cas9prA and the anti-CD3 Ab OKT3. Size exclusion chromatography was performed using a Superose 6 Increase 10/300GL column.

FIG. 11, panels A-C, illustrates Ab-directed targeting of Cas9 RNP to T cells. Panel A: Co-localization of Cas9prA (labeled with Alexa Fluor 488) and T cells after 30 min. Complexation with OKT3 promotes co-localization, as compared to Cas9prA alone or complexed with non-specific IgG. Panel B: Preferential binding of fluorescently-labeled Cas9prA:OKT3 to T cells in a PBMC context. Low background binding of B cells is observed at 30 min. Panel C: Cas9prA:OKT3 induces internalization of the T cell receptor and CD3 after 30 min. as compared to Cas9prA:IgG. Similar internalization is observed with OKT3 alone (not shown).

### DETAILED DESCRIPTION

CRISPR/Cas9, is an RNA-guided targeted genome editing tool that allows genetic “editing” with precision previously unavailable. CRISPR/Cas9 facilitates, inter alia, gene knockouts, knockin SNPs, insertions, and deletions in cell lines and animals. The CRISPR/Cas9 genome editing system typically utilizes two components, Cas9, the endonuclease, and a guide RNA (sgRNA). The guide RNA guides the Cas endonuclease to a specific location in the target genome. With a protospacer-adjacent motif (PAM, e.g., the sequence NGG) present at the 3′ end, the Cas9 endonuclease unwinds the target DNA duplex and cleaves both strands upon recognition of a target sequence by the guide RNA thereby permitting modification of the target genome.

Cas effectors (endonucleases) are frequently delivered to cells as ribonucleoproteins (protein+guide RNA) by electroporation. This ex vivo approach requires removing cells from the body, shocking them, and then re-implanting after gene editing. Alternatively, CRISPR/Cas systems are introduced into cells as vectors (e.g., AAV) comprising nucleic acid constructs that encode the Case endonuclease and guide RNA. Transfection of cells using such constructs, however, is typically quite inefficient.

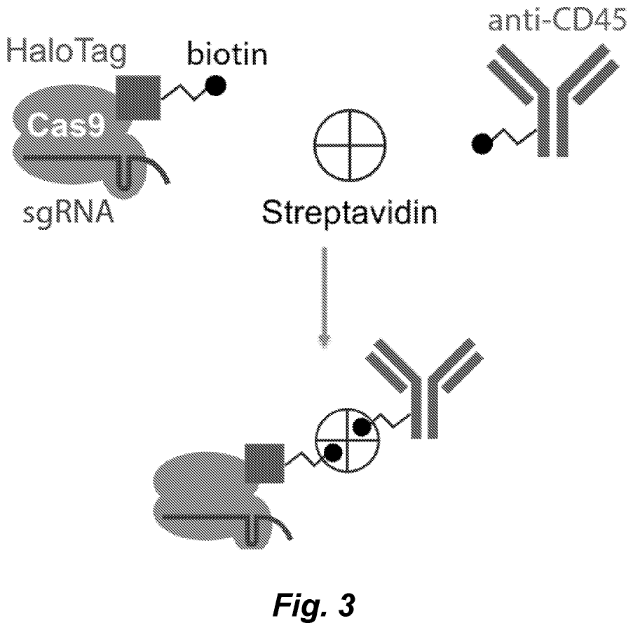

In various embodiments methods and compositions are provided that delivery of Cas effector RNPs to cells without electroporation. In particular, it was a surprising discovery that antibody-mediated uptake of Cas effectors complexed with guide RNA can specifically edit cells expressing high levels of a receptor (target)) for the antibody over cells with low levels of the antibody receptor. This is accomplished using a construct comprising an antibody attached to a Cas endonuclease complexed with a guide RNA (see, e.g., FIG. 3). It is also believed that numerous other targeting moieties in addition to antibody (as described herein) can be effective to deliver a Case effector (e.g., Cas9 complexed with a guide RNA) into a target cell.

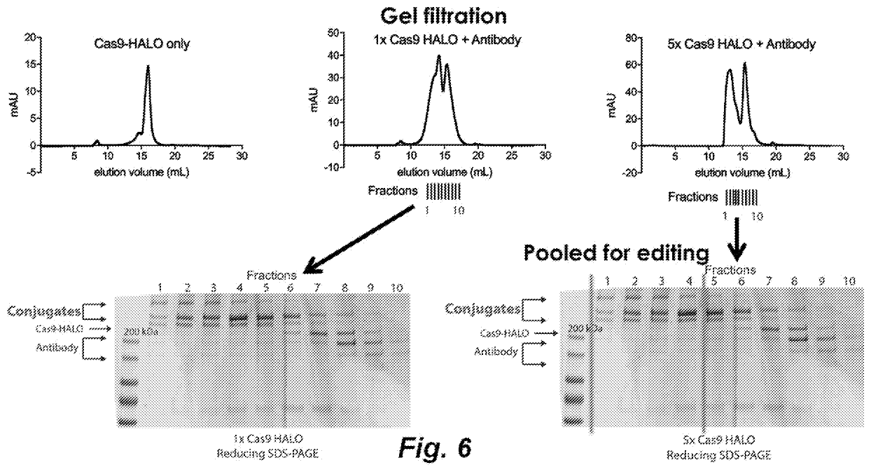

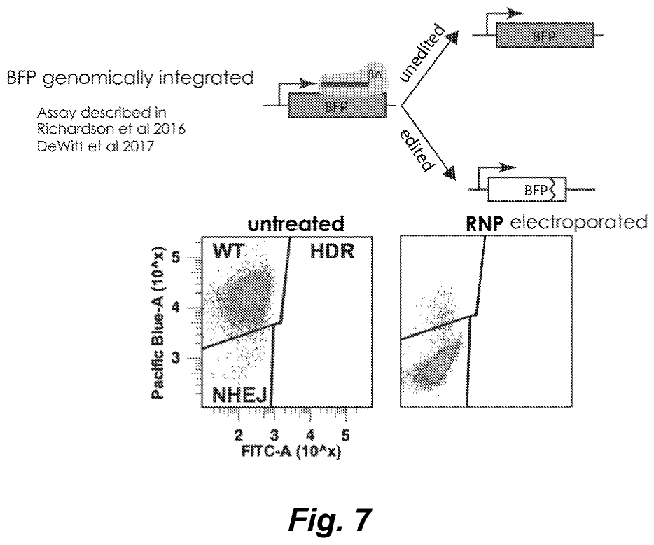

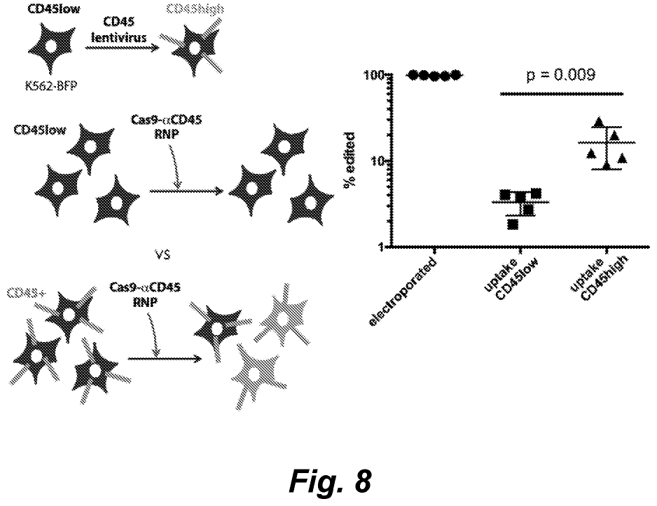

As shown in FIG. 6, the antibody-Cas endonuclease complex is quite stable. Moreover, it was demonstrated that the antibody-directed Cas endonuclease is effective to perform gene editing on target cells (see, e.g., FIG. 7). Additionally, gene editing was preferential for cells expressing the antibody target at high levels (see, e.g., FIG. 8).

This approach could be transformative for in vivo editing. In particular, in view of the teachings provided herein it is believed that antibody-directed Cas effectors (e.g., complexed with a guide RNA) can be delivered in situ and target to a particular tissue or cell type of interest. This approach can be further enhanced with reagents to improve cell penetration or increase endosomal escape. By coupling Cas-guide RNA complexes to different antibodies or other (e.g., synthetic) cell-surface targeting molecules, one can target a wide variety of cells.

The ability to deliver gene editing reagents in situ and have them home to specific tissues is a transformative advance for the treatment of genetic disease. Even in cases where ex vivo editing could be used (e.g. sickle cell disease). it would be far preferable to perform in situ editing. Additionally, several genetic diseases can only be cured through in situ editing due to the inability to remove the target cells or tissue (e.g., lungs, brain, etc.). In situ homing of gene editing reagents could also be extremely useful non-genetic diseases. For example, in situ editing of T cells could be a huge advance for immuno-oncology.

Accordingly, in certain embodiments, constructs for performing gene editing in a mammalian cell are provide where the construct comprises a targeting moiety that binds a cell surface marker (e.g., a receptor), where said targeting moiety (e.g., an antibody, an aptamer, an anticalin, a lectin, a DARPIN, etc.) is attached to a complex comprising a class 2 CRISPR/Cas endonuclease complexed with a corresponding CRISPR/Cas guide RNA that hybridizes to a target sequence within the genomic DNA of the cell (see, e.g., FIG. 3). In certain embodiments, the targeting moiety can be attached to the Cas endonuclease via an avidin/streptavidin linkage. In certain embodiments, where the targeting moiety comprises a protein (e.g., the targeting moiety comprise an antibody or portion thereof), the targeting moiety can be provided as a fusion protein with the Cas endonuclease where the targeting moiety is attached to the Cas endonuclease directly, or through an amino acid, or through a peptide linker. In certain embodiments, the targeting moiety is chemically conjugated to the Case endonuclease (e.g., via a cleavable or non-cleavable linker).

Also provided are pharmaceutical formulations comprising the constructs described herein and a pharmaceutically acceptable carrier or excipient. Additionally, methods of use of the constructs or the pharmaceutical formulations are provided where the methods utilize the construct to edit a target genome in a cell in situ or ex vivo.

Various illustrative targeting moieties and Cas endonucleases as well as methods of attaching the two to provide the constructs are described below.

### Targeting Moieties.

The constructs described herein comprise a targeting moiety that binds a cell surface marker, where said targeting moiety is attached to a complex comprising a class 2 CRISPR/Cas endonuclease complexed with a corresponding CRISPR/Cas guide RNA that hybridizes to a target sequence within the genomic DNA of the cell. In various embodiments, the targeting moiety can include any moiety capable of binding to a cell surface marker. Illustrative cell surface markers include but are not limited to cell surface receptors. Illustrative cell surface markers include, but are not limited to CD45, CD3, erbB2, Her2, CD22, CD74, CD19, CD20, CD33, CD40, MUC1, IL-15R, HLA-DR, EGP-1, EGP-2, G250, prostate specific membrane antigen (PSMA), prostate specific antigen (PSA), prostatic acid phosphatase (PAP), placental alkaline phosphatase, and the like. In certain embodiments, the marker comprises CD45. In certain embodiments, the marker comprises CD3. In certain embodiments, the targeting moiety comprise a moiety that specifically binds to the cell surface marker.

Illustrative binding moieties include, but are not limited to a DNA aptamer, an RNA aptamer, a peptide aptamer, an anticalin, a lectin, a DARPIN, an antibody, and the like.

Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers offer molecular recognition properties that rival that of antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

Methods of aptamer selection/preparation are well known to those of skill in the art. Moreover the process in vitro selection has been automated (see, e.g., Cox and Ellington (2001) Bioorganic & Med. Chem. 9(10): 2525-2531; Cox et al. (2002) Comb. Chem. High Throughput Screen. 5(4): 289-299; Cox et al. (2002) Nucl. Acids Res. 30(20): e108) reducing the duration of a selection experiment from six weeks to three days.

Both DNA and RNA aptamers show robust binding affinities for various targets (see, e.g., Neves et al. (2010) Biophys. Chem. 153(1): 9-16; Baugh et al. (2000) J. Mol. Biol. 301(1): 117-128; Dieckmann et al. (1995) J. Cell. Biol. 59:56-56). DNA and RNA aptamers have been selected for the same target. Lately, a concept of smart aptamers, and smart ligands in general, has been introduced in which aptamers are selected with pre-defined equilibrium (K_{d}) rate (k_{off}/kₒₙ), and thermodynamic (ΔH, ΔS) parameters of aptamer-target interaction. Kinetic capillary electrophoresis is the technology used for the selection of smart aptamers. It obtains aptamers in a few rounds of selection.

Peptide aptamers (Colas et al. (1996) Nature, 380: 548-550) are artificial proteins selected or engineered to bind specific target molecules. These proteins typically consist of one or more peptide loops of variable sequence displayed by a protein scaffold. They are typically isolated from combinatorial libraries and often subsequently improved by directed mutation or rounds of variable region mutagenesis and selection. In vivo, peptide aptamers can bind cellular protein targets. In certain embodiments the peptides that form the aptamer variable regions are synthesized as part of the same polypeptide chain as the scaffold and are constrained at their N and C termini by linkage to it. This double structural constraint decreases the diversity of the conformations that the variable regions can adopt (Spolar et al. (1994) Science, 263: 777-784), and this reduction in conformational diversity lowers the entropic cost of molecular binding when interaction with the target causes the variable regions to adopt a single conformation. As a consequence, peptide aptamers can bind their targets tightly, with binding affinities comparable to those shown by antibodies (nanomolar range).

Peptide aptamer scaffolds are typically small, ordered, soluble proteins. The first scaffold, which is still widely used, is Escherichia coli thioredoxin, the trxA gene product (TrxA) (see, e.g., Colas et al. (1996) Nature, 380: 548-550; Reverdatto et al. (2015) Curr. Top. Med. Chem. 15: 1082-1101). In these molecules, a single peptide of variable sequence is displayed instead of the Gly-Pro motif in the TrxA-Cys-Gly-Pro-Cys-(SEQ ID NO:3) active site loop. Improvements to TrxA include substitution of serines for the flanking cysteines, that prevents possible formation of a disulfide bond at the base of the loop, introduction of a D26A substitution to reduce oligomerization, and optimization of codons for expression in particular cells.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB (see, e.g., Huang et al. (2011) Nucl. Acids Res. 40(1): D271-277).

The affimer protein, an evolution of peptide aptamers, is a small, highly stable protein engineered to display peptide loops that provides a high affinity binding surface for a specific target protein. It is a protein of low molecular weight, 12-14 kDa, [36] derived from the cysteine protease inhibitor family of cystatins (see, e.g., Woodman et al. (2005)J Mol Biol. 352: 1118-1133; Hoffmann et al. (2010) PEDS, 23(5): 403-413; Stadler et al. (2011) PEDS, 24(9): 751-763; Tiede et al. (2014) PEDS, 27(5): 145-155).

The affimer scaffold is a stable protein based on the cystatin protein fold. It displays two peptide loops and an N-terminal sequence that can be randomized to bind different target proteins with high affinity and specificity similar to antibodies. Stabilization of the peptide upon the protein scaffold constrains the possible conformations which the peptide may take, thus increasing the binding affinity and specificity compared to libraries of free peptides.

Other protein scaffolds include, but are not limited to designed ankyrin repeat proteins (DARPins) are a class of non-immunoglobulin proteins that can offer advantages over antibodies for target binding (see, e.g., Stumpp and Amstutz (2007) Curr. Opin. Drug Discov. Devel. 10(2): 153-159). DARPins have been successfully used, for example, for the inhibition of kinases, proteases and drug-exporting membrane proteins. DARPins specifically targeting the cell surface markers (e.g., HER2) also been generated and were shown to function in both in vitro diagnostics and in vivo tumor targeting. DARPins are useful because of their favorable molecular properties, including small size and high stability. The low-cost production in bacteria and the rapid generation of many target-specific DARPins make the DARPin well suited for targeting moieties for essentially any desired target. Additionally, DARPins can be easily generated in multispecific formats, offering the potential to target an effector DARPin to a specific organ or to target multiple receptors with one molecule composed of several DARPins.

Anticalins are another class of engineered ligand-binding proteins that are based on the lipocalin scaffold (see, e.g., Schlehuber and Skerra (2005) Expert. Opin. Biol. Ther. 5(11): 1453-1462). The lipocalin protein architecture is characterized by a compact, rigid β-barrel that supports four structurally hypervariable loops. These loops form a pocket for the specific complexation of differing target molecules. Natural lipocalins occur in human plasma and body fluids, where they usually function in the transport of vitamins, steroids or metabolic compounds. Using targeted mutagenesis of the loop region and biochemical selection techniques, variants with novel ligand specificities, both for low-molecular weight substances and for macromolecular protein targets, can be generated. Due to their small size, typically between 160 and 180 residues, robust tertiary structure and composition of a single polypeptide chain, such “anticalins” can provide several advantages over antibodies concerning economy of production, stability during storage, faster pharmacokinetics and better tissue penetration.

In certain embodiments the targeting moieties attached to the Cas endonuclease/guide RNA complex comprise antibodies. In certain embodiments the antibodies are monoclonal antibodies. Such antibodies include full length immunoglobulins (e.g., IgG, IgA, IgM, etc.) as well as antibody fragments including, but are not limited to, Fab, Fab′, Fab′-SH, F(ab′)₂, Fv, Fv′, Fd, Fd′, scFv, hsFv fragments, single-chain antibodies, cameloid antibodies, diabodies, and the like. Methods of producing such antibodies are well known to those of skill in the art. Such antibodies are commercially available (see, e.g., from Pacific Immunology, Ramona Calif., ABClonal, Woburn, Mass., etc.).

In certain embodiments the antibody targeting moieties can be constructed as unibodies. UniBody technology is an antibody technology that produces a stable, smaller antibody format with an anticipated longer therapeutic window than certain small antibody formats. In certain embodiments unibodies are produced from IgG4 antibodies by eliminating the hinge region of the antibody. Unlike the full size IgG4 antibody, the half molecule fragment is very stable and is termed a uniBody. Halving the IgG4 molecule leaves only one area on the UniBody that can bind to a target. Methods of producing unibodies are described in detail in PCT Publication WO2007/059782, which is incorporated herein by reference in its entirety (see, also, Kolfschoten et al. (2007) Science 317: 1554-1557).

In certain embodiments the antibody targeting moieties can be constructed affibody molecules. Affibody molecules are class of affinity proteins based on a 58-amino acid residue protein domain, derived from one of the IgG-binding domains of staphylococcal protein A. This three helix bundle domain has been used as a scaffold for the construction of combinatorial phagemid libraries, from which affibody variants that target the desired molecules can be selected using phage display technology (see, e.g., Nord et al. (1997) Nat. Biotechnol. 15: 772-777; Ronmark et al. (2002) Eur. J. Biochem., 269: 2647-2655). Details of Affibodies and methods of production are known to those of skill (see, e.g., U.S. Pat. No. 5,831,012 which is incorporated herein by reference in its entirety).

In certain embodiments the antibodies used for targeting moieties are internalizing antibodies. Methods of producing internalizing antibodies, e.g., from phage display libraries, are well known to those of skill in the art (see, e.g., Nielsen et al. (2000) Pharmaceut. Sci. Technol. Today, 3(8): 282-291) Zhou and Marks (2012) Meth. Enzym. 502: 43-66; and the like).

### Attaching the Antibody to the Cas Polypeptide.

Methods of coupling the Cas effector (e.g., a complex comprising a class 2 CRISPR/Cas endonuclease and a guide RNA) to the targeting moiety are well known to those of skill in the art. Examples include, but are not limited to the use of biotin and avidin or streptavidin (see, e.g., U.S. Pat. No. 4,885,172 A), typical biotin/avidin alternatives (e.g., FITC/anti-FITC (see, e.g., Harmer and Samuel (1989) J. Immunol. Meth. 122(1): 115-221), dioxigenin/anti-dioxigenin, and the like), by traditional chemical conjugation using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like. In certain embodiments, where the targeting moiety comprises a polypeptide, the Cas endonuclease (Cas effector) can be expressed as a fusion protein with the targeting moiety. In such instances, the fusion can be directly between the Cas endonuclease, or through an intervening amino acid, or through a peptide linker. In certain embodiments the peptide linker, when present, can be an enzymatically cleavable peptide linker.

As noted above, in certain embodiments the targeting moiety (e.g., antibody, lectin, aptamer, anticaline, lectin, DarPIN) is attached to the Cas effector (e.g., cas endonuclease via a linker (linking agent). A “linker” or “linking agent” as used herein, is a molecule that is used to join two or more molecules. In certain embodiments, the linker is typically capable of forming covalent bonds to both molecule(s) (e.g., the targeting moiety and the Cas endonuclease). Suitable linkers are well known to those of skill in the art and include, but are not limited to, straight or branched-chain carbon linkers, heterocyclic carbon linkers, or peptide linkers. In certain embodiments, the linkers can be joined to the constituent amino acids through their side groups (e.g., through a disulfide linkage to cysteine) as noted above, while in other embodiments, the linkers will be joined to the alpha carbon amino and carboxyl groups of the terminal amino acids when such are present.

Typically the linker comprises a functional group that is reactive with a corresponding functional group on the targeting moiety and/or Cas endonuclease. A bifunctional linker has one functional group reactive with a group on the targeting moiety (e.g., antibody) and another functional group reactive on the Cas endonuclease and can be used to form the desired conjugate. A heterobifunctional linker typically comprises two or more different reactive groups that react with sites on the targeting moiety and on the Cas endonuclease, respectively. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups.

Such reactions and functional groups are illustrative and non-limiting. Other illustrative suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Chemical Conjugation.

In certain embodiments, the targeting moiety (e.g., an anti-CD3 antibody, an anti-CD45 antibody, etc.) is chemically conjugated to the Cas effector (e.g., Cas endonuclease). Means of chemically conjugating molecules are well known to those of skill.

The procedure for conjugating two molecules varies according to the chemical structure of the moieties to be joined. Polypeptides typically contain a variety of functional groups; e.g., carboxylic acid (COOH) or free amine (—NH₂) groups that are available for reaction with a suitable functional group on the other peptide, or on a linker to join the molecules thereto.

For example, a common approach to the conjugation of antibodies (or other polypeptide targeting moieties), can involve the use of available lysines or reduced cysteine disulfides to form the conjugates. Lysine and cysteine as natural amino acids frequently exist in the antibodies and are readily available for reaction. For example, the thiol groups produced from reduction of cystines and primary amino group of lysines can be directly exploited. In certain illustrative, but non-limiting embodiments the primary amine in the lysines is easily reacted with N-hydroxysuccinimide (NHS) esters linker to form stable amide bonds and a great number of commercial linkers depend on this method. In certain embodiments, the amine of lysine can also be used to make an amidine with a pendant thiol for connection to a linker or payload via 2-imiothiolane (Traut's reagent).

In another illustrative, but non-limiting example, cysteins, as natural amino acids in the targeting moieties (e.g., antibodies) can be tethered through disulfide bridges. Under appropriate conditions, the disulfide bonds can be selectively reduced by the DL-Dithiothreitol (DTT) or Tris(2-carboxyethyl)phosphine (TCEP) and provide reactive thiol groups. The free thiol groups as attachment sites on the antibodies can be conjugated with a small linker molecule through different chemical reactions, such as Michael additions, a-halo carbonyl alkylations and disulfide formation. The hydrolyzed succinimide-thioether linker is a common useful linkage.

In certain embodiments the antibodies can include genetically encoded unnatural amino acids to provide linkage sites. Commonly utilized unnatural amino acids in the targeting moieties can include, inter alia, para-acetyl Phe, para-azido Phe, propynyl-Tyr, and the like.

In certain embodiments, the linker comprises a cleavable linker. Cleavable linkers include both chemically cleavable linkers and enzymatically cleavable linkers.

A number of different chemically cleavable linkers are known to those of skill in the art (see, e.g., U.S. Pat. Nos. 4,618,492; 4,542,225, and 4,625,014). Illustrative chemically cleavable linkers include, but are not limited to, acid-labile linkers, disulfide linkers, and the like. Acid-labile linkers are designed to be stable at pH levels encountered in the blood, but become unstable and degrade when the low pH environment in lysosomes is encountered. Acid-sensitive linkers include, but are not limited to hydrazones, acetals, cis-aconitate-like amides, and silyl ethers (see, e.g., Perez et al. (2013) Drug Discov. Today, 1-13). Hydrazones are easily synthesized and have a plasma half-life of 183 hours at pH 7 and 4.4 hours at pH 5, indicating that they are selectively cleavable under acidic conditions such as those found in the lysosome (see, e.g., Doronina et al. 92013) Nat. Biotechnol. 21(7): 778-784).

Disulfide bridges are cleavable linkers that take advantage of the cellular reducing environment (see, e.g., Saito et al. (2013) Adv. Drug Deliv. Rev. 55(2): 199-215). After internalization and degradation, disulfide bridges can release drugs in the lysosome.

Enzymatically cleavable linkers are selected to be cleaved by an enzyme (e.g., a protease). Protease-cleavable linkers are typically designed to be stable in blood/plasma, but rapidly release free drug inside lysosomes in target cells upon cleavage by lysosomal enzymes. In various embodiments, they can take advantage of the high levels of protease activity inside lysosomes. The most popular enzymatic cleavage sequence is the dipeptide valine-citrulline, combined with a self-immolative linker p-aminobenzyl alcohol (PAB). Cleavage of an amide-linked PAB triggers a 1,6-elimination of carbon dioxide and concomitant release of the free drug in parent amine form (see, e.g., Burke et al. (2009) Bioconjug. Chem. 20(6): 1242-1250).

A library of dipeptide linkers was screened by Debowchik and co-workers to measure the rate of doxorubicin release by enzymatic hydrolysis (see, e.g., Dubowchik et al. (2002) Bioconjug. Chem. 13(4): 855-869; Dubowchik et al. (2002) Bioorg. Med. Chem. Lett. 12(11): 1529-1532). They found that Phe-Lys was cleaved most rapidly with a half-life of 8 min, followed closely by Val-Lys with a half-life of 9 min. In stark contrast, Val-Cit showed a half-life of 240 min. They also found that removal of the PAB group reduced the cleavage rate, presumably through steric interference with enzyme binding.

Another study compared the potency of auristatin derivative MMAE linked by dipeptide linkers Phe-Lys and Val-Cit and an analogous hydrazone linker. The Val-Cit linker proved to be over 100 times as stable as the hydrazone linker in human plasma. Most significantly, the Phe-Lys linker was substantially less stable than Val-Cit in human plasma, which accounts for its current popularity (see, e.g., Doronina et al. (2003) Nat. Biotechnol. 21(7): 778-784).

Non-peptide enzymatically cleavable linkers are also known to those of skill in the art. A glucuronide linker incorporates a hydrophilic sugar group that is cleaved by the lysosomal enzyme beta glucuronidase. Once the sugar is cleaved from the phenolic backbone, self-immolation of the PAB group releases the conjugated moiety (see, e.g., Jeffrey et al. (3006) Bioconjug. Chem. 17(3): 831-840).

In certain embodiments, the linker used to join an antibody to a Cas effector (e.g., a complex comprising a class 2 CRISPR/Cas endonuclease and a guide RNA) comprises a protein that binds (e.g., non-covalently binds to the antibody (e.g., to the Fc region of the antibody). A number of bacterial proteins are known to bind mammalian immunoglobins and include, but are not limited to, protein A, G, L, Z, and recombinant (fusion proteins) derivatives thereof (see, e.g., Table 1; Rodrigo et al. (2015) Antibodies, 4: 259-277; Konrad et al. (2011) Bioconjug. Chem. 22: 2395-2403; Kihlberg et al. (1996) Eur. J. Biochem. 240: 556-563; Nilsson et al. (1987) Protein Eng. Des. Sel. 1: 107-113; Ghitescu et al. (1991) J. Histochem. Cytochem. 39: 1057-1065; Akerstrom and Bjorck (1986) J. Biol. Chem. 261: 10240-10247; Svensson et al. (1998) Eur. J. Biochem. 258: 890-896)

TABLE 1
Illustrative proteins that can be incorporated into linkers to
bind cell targeting moieties (e.g., cell-targeting antibodies).

| Protein Used | |
|---|---|
| in Linkage | Reference |
| Protein A | Ey et al. (1978) Immunochemistry, 15: 429-436; |
| | Svensson et al. (1998) Eur. J. Biochem. 258: 890-896 |
| Protein G | Akerstrom et al. (1986) J. Biol. Chem. |
| | 261: 10240-10247 |
| Protein L | Rodrigo et al. (2015) Antibodies, |
| | 4: 259-277 |
| Protein Z | Konrad et al. (2011) Bioconjug. Chem. |
| | 22: 2395-2403 |
| Protein LG | Kihlberg et al. (1996) Eur. J. Biochem. |
| | 240: 556-563 |
| Protein LA | Nilsson et al. (1987) Protein Eng. Des. Sel. |
| | 1: 107-113 |
| Protein AG | Ghitescu et al. (1991) J. Histochem. Cytochem. |
| | 39: 1057-1065 |

A number of cyclic peptides are known that bind to antibody constant regions and can be used to link antibodies to the Cas effector. Examples of such peptides include, but are not limited to PAM (Fassina et al. (2006) J. Mol. Recognit. 9: 564-569), D-PAM (Verdoliva et al. (2002) J. Immunol. Meth. 271: 77-88), D-PAM-θ (Dinon et al. (2011) J. Mol. Recognit. 24: 1087-1094), TWKTSRISIF (SEQ ID NO:4) and FGRLVSSIRY (SEQ ID NO:5, Krook et al. (1998) J. Immunol. Meth. 221: 151-157), Fc-III (DeLano et al. (2000) Science, 287: 1279-1283), EPIHRSTLTALL (SEQ ID NO:6, Ehrlich et al. (2001) J. Biochem. Biophys. Meth. 49: 443-454), HWRGWV (SEQ ID NO:7, Yang et al. (2006) J. Peptide Res. 66: 110-137), HYFKFD (SEQ ID NO:8, Yang et al. (2009) J. Chromatogr. A, 1216: 910-918), HFRRHL (SEQ ID NO:9, Menegatti et al. (2016) J. Chromatogr. A, 1445: 93-104), NKFRGKYK (SEQ ID NO:10) and NARKFYKG (SEQ ID NO:11, Sugita et al. (2013) Biochem. Eng. J. 79: 33-40), KHRFNKD (SEQ ID NO:12, Yoo and Choi (2015) BioChip J. 10: 88-94), and the like (see, e.g., Choe et al. (2016) Materials, 9: 994).

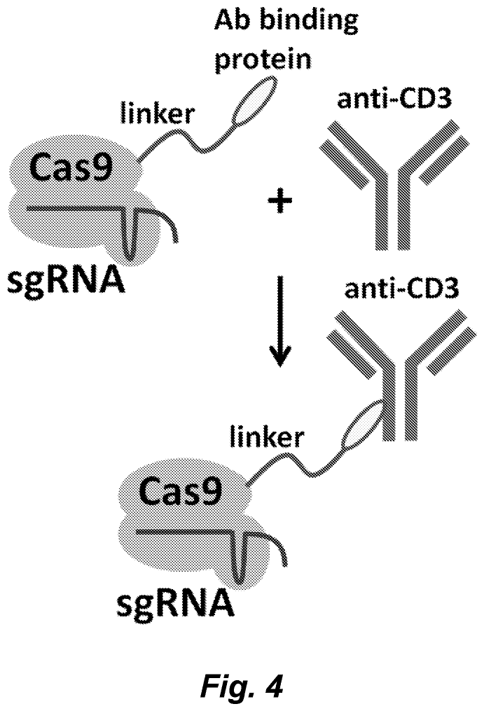

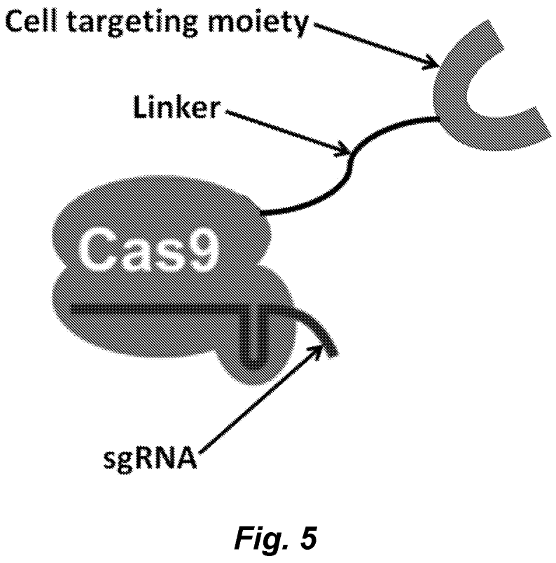

In certain embodiments the antibody-binding protein (peptide) is attached to the Cas endonuclease by a linker (see, e.g., FIG. 4). In certain embodiments the linker attaching the antibody binding protein to the Cas endonuclease comprises a cleavable linker or a non-cleavable linker described herein.

In certain embodiments the Cas effector is linked to a targeting moiety by a linker comprising a peptide that binds to an antibody (e.g., to Fc region of an antibody) at high pH, but releases the antibody at lower pH. In certain embodiments, the peptide comprises the FcB6.1 peptide (see, e.g., Strauch et al. (2014) Proc. Natl. Acad. Sci. USA, 111(2): 675-680.

Many procedures and linker molecules for attachment of various molecules to peptides or proteins are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075). Illustrative non-peptide linkers suitable for chemical conjugation are shown in Table 2.

Fusion Proteins.

In certain embodiments where targeting moiety comprise a polypeptide (e.g., is an antibody or other binding protein) the peptide can be fused directly to the Cas endonuclease, fused through an amino acid, or fused through a peptide linker. In certain embodiments the targeting moiety attached to the Cas endonuclease is simply synthesized directly using methods of chemical peptide synthesis.

In certain embodiments, the targeting moiety attached to the Cas endonuclease can be recombinantly expressed as a fusion protein (e.g., directly fused, joined through an amino acid, or joined through a linker). Generally this involves creating a DNA sequence that encodes the fusion protein, placing the DNA in an expression cassette under the control of a particular promoter, expressing the protein in a host, isolating the expressed protein and, if required, renaturing the protein.

DNA encoding the fusion proteins can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This can be converted into double stranded DNA by hybridization with a complementary sequence or by polymerization with a DNA polymerase using the single strand as a template. One of skill would recognize that while chemical synthesis of DNA is limited to sequences of about 100 bases, longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

In certain embodiments, DNA encoding fusion proteins of the present invention may be cloned using DNA amplification methods such as polymerase chain reaction (PCR). Thus, for example, the therapeutic moiety “D” can PCR amplified, using a sense primer containing the restriction site for NdeI and an antisense primer containing the restriction site for HindIII. This produces a nucleic acid encoding the targeting moiety and having terminal restriction sites. Similarly the Cas endonuclease and/or CasP-L (where L is an amino acid or a peptide linker) can be provided having complementary restriction sites. Ligation of sequences and insertion into a vector produces a vector encoding the fusion protein.

As noted above, while the targeting moiety and Cas endonuclease can be directly joined together, one of skill will appreciate that they can be separated by linker consisting of one or more amino acids. Generally the spacer will have no specific biological activity other than to join the proteins or to preserve some minimum distance or other spatial relationship between them. However, the constituent amino acids of the spacer may be selected to influence some property of the molecule such as the folding, net charge, or hydrophobicity. In certain embodiments the linker may comprise an enzymatic cleavage site.

The nucleic acid sequences encoding the fusion proteins can be expressed in a variety of host cells, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion proteins can be purified according to standard procedures of the art, including ammonium sulfate precipitation, affinity columns, column chromatography, gel electrophoresis and the like (see, generally, R. Scopes (1982) Protein Purification, Springer-Verlag, N.Y.; Deutscher (1990) Methods in Enzymology Vol. 182: Guide to Protein Purification., Academic Press, Inc. N.Y.). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, the fusion protein may possess a conformation substantially different than the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem., 268: 14065-14070; Kreitman and Pastan (1993) Bioconjug. Chem., 4: 581-585; and Buchner, et al. (1992) Anal. Biochem., 205: 263-270).

One of skill would recognize that modifications can be made to the fusion proteins without diminishing their biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids placed on either terminus to create conveniently located restriction sites or termination codons.

As indicated above, in various embodiments an amino acid, or a peptide linker is used to join the targeting moiety to the Cas endonuclease. In various embodiments the peptide linker is relatively short, typically about 20 amino acids or less or about 15 amino acids or less or about 10 amino acids or less or about 8 amino acids or less or about 5 amino acids or less or about 3 amino acids or less, or is a single amino acid. Suitable illustrative linkers include, but are not limited to the amino acids or peptide linkers shown in Table 2.

TABLE 2
Illustrative peptide and non-peptide linkers.

| | SEQ |
|---|---|
| | ID |
| Linker | NO: |
| A | |
| R | |
| N | |
| D | |
| B | |
| C | |
| E | |
| Q | |
| Z | |
| G | |
| H | |
| I | |
| L | |
| K | |
| M | |
| F | |
| P | |
| S | |
| T | |
| W | |
| Y | |
| V | |
| G | |
| AAA | |
| GGG | |
| SGG | |
| SAT | |
| PYP | |
| ASA | |
| GGGG | 13 |
| PSGSP | 14 |
| PSPSP | 15 |
| PSPSP | 16 |
| KKKK | 17 |
| RRRR | 18 |
| ASASA | 19 |
| GGSGGS | 20 |
| GGGGS | 21 |
| GGGGS GGGGS | 22 |
| GGGGS GGGGS GGGGS | 23 |
| GGGGS GGGGS GGGGS GGGGS | 24 |
| GGGGS GGGGS GGGGS GGGGS GGGGS | 25 |
| GGGGS GGGGS GGGGS GGGGS GGGGS GGGGS | 26 |
| GGGGS GGGGS GGGGS FK GGGGS GGGGS | 27 |
| GGGGS | |
| GGGGS GGGGS GGGGS VA GGGGS GGGGS | 28 |
| GGGGS | |
| 2-nitrobenzene or O-nitrobenzyl | |
| Nitropyridyl disulfide | |
| Dioleoylphosphatidylethanolamine(DOPE) | |
| S-acetylmercaptosuccinic acid | |
| 1,4,7,10-tetraazacyclododecane-1,4,7,10-tetracetic | |
| acid(DOTA) | |
| β-glucuronide and 3-glucuronide variants | |
| Poly (alkylacrylic acid) | |
| Benzene-based linkers(for example: 2,5-Bis(hexyloxy)- | |
| 1,4-bis[2,5-bis(hexyloxy)-4-formyl- | |
| phenylenevinylene]benzene) and like molecules | |
| Disulfide linkages | |
| Poly(amidoamine) or like dendrimers linking multiple | |
| target and killing peptides in one molecule | |
| Hydrazone and hydrazone variant linkers | |
| PEG of any chain length | |
| Succinate, formate, acetate butyrate, other like organic | |
| acids | |
| Aldols, alcohols, or enols | |
| Peroxides | |
| alkane or alkene groups of any chain length | |
| Variants of any of the above linkers containing halogen | |
| or thiol groups | |
| Quaternary-ammonium-salt linkers | |
| Allyl(4-methoxyphenyl)dimethylsilane | |
| 6-(Allyloxycarbonylamino)-1-hexanol | |
| 3-(Allyloxycarbonylamino)-1-propanol | |
| 4-Aminobutyraldehyde diethyl acetal | |
| (E)-N-(2-Aminoethyl)-4-{2-[4-(3- | |
| azidopropoxy)phenyl]diazenyl}benzamide hydrochloride | |
| N-(2-Aminoethyl)maleimide trifluoroacetate | |
| Amino-PEG4-alkyne | |
| Benzyl N-(3-hydroxypropyl)carbamate | |
| 4-(Boc-amino)-1-butanol | |
| 4-(Boc-amino)butyl bromide | |
| 2-(Boc-amino)ethanethiol | |
| 2-[2-(Boc-amino)ethoxy]ethoxyacetic acid | |
| (dicyclohexylammonium)salt | |
| 2-(Boc-amino)ethyl bromide | |
| 6-(Boc-amino)-1-hexanol | |
| 21-(Boc-amino)-4,7,10,13,16,19-hexaoxaheneicosanoic | |
| acid | |
| 6-(Boc-amino)hexyl bromide | |
| 5-(Boc-amino)-1-pentanol | |
| 3-(Boc-amino)-1-propanol | |
| 3-(Boc-amino)propyl bromide | |
| 15-(Boc-amino)-4,7,10,13-tetraoxapentadecanoic acid | |
| N-Boc-1,4-butanediamine | |
| N-Boc-cadaverine | |
| N-Boc-ethanolamine | |
| N-Boc-ethylenediamine | |
| N-Boc-2,2′-(ethylenedioxy)diethylamine | |
| N-Boc-1,6-hexanediamine | |
| N-Boc-1,6-hexanediamine hydrochloride | |
| N-Boc-4-isothiocyanatoaniline | |
| N-Boc-4-isothiocyanatobutylamine | |
| N-Boc-2-isothiocyanatoethylamine | |
| N-Boc-3-isothiocyanatopropylamine | |
| N-Boc-N-methylethylenediamine | |
| N-Boc-m-phenylenediamine | |
| N-Boc-p-phenylenediamine | |
| 2-(4-Boc-1-piperazinyl)acetic acid | |
| N-Boc-1,3-propanediamine | |
| N-Boc-1,3-propanediamine | |
| N-Boc-N′-succinyl-4,7,10-trioxa-1,13-tridecanediamine | |
| N-Boc-4,7,10-trioxa-1,13-tridecanediamine | |
| N-(4-Bromobutyl)phthalimide | |
| 4-Bromobutyric acid | |
| 4-Bromobutyryl chloride purum | |
| 4-Bromobutyryl chloride | |
| N-(2-Bromoethyl)phthalimide | |
| 6-Bromo-1-hexanol | |
| 3-(Bromomethyl)benzoic acid N-succinimidylester | |
| 4-(Bromomethyl)phenyl isothiocyanate | |
| 8-Bromooctanoic acid | |
| 8-Bromo-1-octanol | |
| 4-(2-Bromopropionyl)phenoxyacetic acid | |
| N-(3-Bromopropyl)phthalimide | |
| 4-(tert-Butoxymethyl)benzoic acid | |
| tert-Butyl 2-(4-{[4-(3- | |
| azidopropoxy)phenyl]azo}benzamido)ethylcarbamate | |
| 2[2-(tert-Butyldimethylsilyloxy)ethoxy]ethanamine | |
| tert-Butyl 4-hydroxybutyrate | |
| 4-(2-Chloropropionyl)phenylacetic acid | |
| 1,11-Diamino-3,6,9-trioxaundecane | |
| di-Boc-cystamine | |
| Diethylene glycol monoallyl ether | |
| 3,4-Dihydro-2H-pyran-2-methanol | |
| 4-[(2,4-Dimethoxyphenyl)(Fmoc- | |
| amino)methyl]phenoxyacetic acid | |
| 4-(Diphenylhydroxymethyl)benzoic acid | |
| 4-(Fmoc-amino)-1-butanol | |
| 2-(Fmoc-amino)ethanol | |
| 2-[2-(Fmoc-amino)ethoxy]ethylamine hydrochloride | |
| 2-(Fmoc-amino)ethyl bromide | |
| 6-(Fmoc-amino)-1-hexanol | |
| 5-(Fmoc-amino)-1-pentanol | |
| 3-(Fmoc-amino)-1-propanol | |
| 3-(Fmoc-amino)propyl bromide | |
| N-Fmoc-2-bromoethylamine | |
| N-Fmoc-1,4-butanediamine hydrobromide | |
| N-Fmoc-cadaverine hydrobromide | |
| N-Fmoc-ethylenediamine hydrobromide | |
| N-Fmoc-1,6-hexanediamine hydrobromide | |
| N-Fmoc-1,3-propanediamine hydrobromide | |
| N-Fmoc-N″-succinyl-4,7,10-trioxa-1,13-tridecanediamine | |
| (3-Formyl-1-indolyl)acetic acid | |
| 6-Guanidinohexanoic acid | |
| 4-Hydroxybenzyl alcohol | |
| N-(4-Hydroxybutyl)trifluoroacetamide | |
| 4′-Hydroxy-2,4-dimethoxybenzophenone | |
| N-(2-Hydroxyethyl)maleimide | |
| 4-[4-(1-Hydroxyethyl)-2-methoxy-5- | |
| nitrophenoxy]butyric acid | |
| N-(2-Hydroxyethyl)trifluoroacetamide | |
| N-(6-Hydroxyhexyl)trifluoroacetamide | |
| 4-Hydroxy-2-methoxybenzaldehyde | |
| 4-Hydroxy-3-methoxybenzyl alcohol | |
| 4-(Hydroxymethyl)benzoic acid | |
| 4-(4-Hydroxymethyl-3-methoxyphenoxy)butyric acid | |
| 4-(Hydroxymethyl)phenoxyacetic acid | |
| 3-(4-Hydroxymethylphenoxy)propionic acid | |
| N-(5-Hydroxypentyl)trifluoroacetamide | |
| 4-(4′-Hydroxyphenylazo)benzoic acid | |
| N-(3-Hydroxypropyl)trifluoroacetamide | |
| 2-Maleimidoethyl mesylate technical | |
| 4-Mercapto-1-butanol | |
| 6-Mercapto-1-hexanol | |
| Phenacyl 4-(bromomethyl)phenylacetate | |
| 4-Sulfamoylbenzoic acid | |
| N-Trity1-1,2-ethanediamine hydrobromide | |
| 4-(Z-Amino)-1-butanol | |
| 6-(Z-Amino)-1-hexanol | |
| 5-(Z-Amino)-1-pentanol | |
| N-Z-1,4-Butanediamine hydrochloride | |
| N-Z-Ethanolamine | |
| N-Z-Ethylenediamine hydrochloride | |
| N-Z-1,6-hexanediamine hydrochloride | |
| N-Z-1,5-pentanediamine hydrochloride | |
| N-Z-1,3-Propanediamine hydrochloride | |
| 1,4-Bis[3-(2-pyridyldithio)propionamido]butane | |

BMOE(bis-maleimidoethane)
BM(PEG)2 (1,8-bismaleimido-diethyleneglycol)
BM(PEG)3 (1,11-bismaleimido-triethyleneglycol)
DTME(dithio-bis-maleimidoethane)
BMOE(bis-maleimidoethane)
DTME(dithio-bis-maleimidoethane)

Maleimidoacetic acid N-hydroxysuccinimide ester
4-(N-Maleimidomethyl)cyclohexanecarboxylic acid N-
hydroxysuccinimide ester
4-(N-Maleimidomethyl)cyclohexane-1-carboxylic acid 3-
sulfo-N-hydroxysuccinimide ester
4-(4-Maleimidophenyl)butyric acid N-hydroxysuccinimide ester
hydroxysuccinimide ester
3-(Maleimido)propionic acid N-hydroxysuccinimide ester

(All amino-acid-based linkers could be L, D, combinations of L and D forms, β-form, and the like)

### The CRISPR/Cas System

Compelling evidence has recently emerged for the existence of an RNA-mediated genome defense pathway in archaea and many bacteria that has been hypothesized to parallel the eukaryotic RNAi pathway (for reviews, see Godde and Bickerton (2006) J. Mol. Evol. 62: 718-729; Lillestol et al. (2006) Archaea 2: 59-72; Makarova et al. (2006) Biol. Direct 1: 7; Sorek et al. (2008) Nat. Rev. Microbiol. 6: 181-186). Known as the CRISPR-Cas system or prokaryotic RNAi (pRNAi), the pathway is believed to arise from two evolutionarily and often physically linked gene loci: the CRISPR (clustered regularly interspaced short palindromic repeats) locus, that encodes RNA components of the system, and the cas (CRISPR-associated) locus, that encodes proteins (see, e.g., Jansen et al. (2002) Mol. Microbiol. 43: 1565-1575; Makarova et al., (2002) Nucl. Acids Res. 30: 482-496; Makarova et al. (2006) Biol. Direct 1: 7; Haft et al. (2005) PLoS Comput. Biol. 1: e60). CRISPR loci in microbial hosts contain a combination of CRISPR-associated (Cas) genes as well as non-coding RNA elements capable of programming the specificity of the CRISPR-mediated nucleic acid cleavage. The individual Cas proteins do not share significant sequence similarity with protein components of the eukaryotic RNAi machinery, but have analogous predicted functions (e.g., RNA binding, nuclease, helicase, etc.) (see, e.g., Makarova et al. (2006) Biol. Direct 1: 7). The CRISPR-associated (cas) genes are often associated with CRISPR repeat-spacer arrays. More than forty different Cas protein families have been described. Of these protein families, Cas1 appears to be ubiquitous among different CRISPR/Cas systems. Particular combinations of cas genes and repeat structures have been used to define 8 CRISPR subtypes (E. coli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Type II CRISPR/Cas Endonucleases Cas 9)

In natural Type II CRISPR/Cas systems, Cas9 functions as an RNA-guided endonuclease that uses a dual-guide RNA having a crRNA and trans-activating crRNA (tracrRNA) for target recognition and cleavage by a mechanism involving two nuclease active sites in Cas9 that together generate double-stranded DNA breaks (DSBs), or can individually generate single-stranded DNA breaks (SSBs). The Type II CRISPR endonuclease Cas9 and engineered dual—(dgRNA) or single guide RNA (sgRNA) form a ribonucleoprotein (RNP) complex that can be targeted to a desired DNA sequence. Guided by a dual-RNA complex or a chimeric single-guide RNA, Cas9 generates site-specific DSBs or SSBs within double-stranded DNA (dsDNA) target nucleic acids, that are repaired either by non-homologous end joining (NHEJ) or homology-directed recombination (HDR).

As noted above, in various embodiments, constructs are provided that comprise an antibody (e.g., an internalizing antibody) attached to a type II CRISPR/Cas endonuclease. A type II CRISPR/Cas endonuclease is a type of class 2 CRISPR/Cas endonuclease. In some cases, the type II CRISPR/Cas endonuclease is a Cas9 protein. A Cas9 protein forms a complex with a Cas9 guide RNA. The guide RNA provides target specificity to a Cas9-guide RNA complex by having a nucleotide sequence (a guide sequence) that is complementary to a sequence (the target site) of a target nucleic acid (as described elsewhere herein). The Cas9 protein of the complex provides the site-specific activity. In other words, the Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA

The type II CRISPR, initially described in S. pyogenes, is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences where processing occurs by a double strand-specific RNase III in the presence of the Cas9 protein. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In addition, the tracrRNA must also be present as it base pairs with the crRNA at its 3′ end, and this association triggers Cas9 activity. Finally, Cas9 mediates cleavage of target DNA to create a double-stranded break within the protospacer. Activity of the CRISPR/Cas system typically takes place by: (i) insertion of alien DNA sequences into the CRISPR array to prevent future attacks, in a process called “adaptation”; (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system.

A Cas9 protein can bind and/or modify (e.g., cleave, nick, methylate, demethylate, etc.) a target nucleic acid and/or a polypeptide associated with target nucleic acid (e.g., methylation or acetylation of a histone tail) (e.g., when the Cas9 protein includes a fusion partner with an activity). In some cases, the Cas9 protein is a naturally-occurring protein (e.g., naturally occurs in bacterial and/or archaeal cells). In other cases, the Cas9 protein is not a naturally-occurring polypeptide (e.g., the Cas9 protein is a variant Cas9 protein, a chimeric protein, and the like)

Type II CRISPR systems have been found in many different bacteria. BLAST searches on available genomes by Fonfara et al. ((2013) Nuc Acid Res 42(4):2377-2590) found Cas9 orthologs in 347 species of bacteria. Additionally, this group demonstrated in vitro CRISPR/Cas cleavage of a DNA target using Cas9 orthologs from S. pyogenes, S. mutans, S. therophilus, C. jejuni, N. meningitides, P. multocida and F. novicida. Thus, the term “Cas9” refers to an RNA guided DNA nuclease comprising a DNA binding domain and two nuclease domains, where the gene encoding the Cas9 may be derived from any suitable bacteria.

The typical Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. In certain embodiments the Cas 9 nuclease can be engineered such that only one of the nuclease domains is functional, creating a Cas nickase (see, e.g., Jinek et al. (2012) Science 337:816). Nickases can be generated by specific mutation of amino acids in the catalytic domain of the enzyme, or by truncation of part or all of the domain such that it is no longer functional. Since Cas 9 comprises two nuclease domains, this approach may be taken on either domain. A double strand break can be achieved in the target DNA by the use of two such Cas 9 nickases. The nickases will each cleave one strand of the DNA and the use of two will create a double strand break.

The primary products of the CRISPR loci appear to be short RNAs that contain the invader targeting sequences, and are termed guide RNAs or prokaryotic silencing RNAs (psiRNAs) based on their hypothesized role in the pathway (see, e.g., Makarova et al. (2006) Biol. Direct 1: 7; Hale et al. (2008) RNA, 14: 2572-2579). RNA analysis indicates that CRISPR locus transcripts are cleaved within the repeat sequences to release ˜60- to 70-nt RNA intermediates that contain individual invader targeting sequences and flanking repeat fragments (see, e.g., Tang et al. (2002) Proc. Natl. Acad. Sci. USA, 99: 7536-7541; Tang et al. (2005) Mol. Microbiol. 55: 469-481; Lillestol et al. (2006) Archaea 2: 59-72; Brouns et al. (2008) Science 321: 960-964; Hale et al. (2008) RNA, 14: 2572-2579). In the archaeon Pyrococcus furiosus, these intermediate RNAs are further processed to abundant, stable 35- to 45-nt mature psiRNAs (Hale et al. 2008. RNA, 14: 2572-2579).

The requirement of the crRNA-tracrRNA complex can be avoided by use of an engineered “single-guide RNA” (sgRNA) that comprises the hairpin normally formed by the annealing of the crRNA and the tracrRNA (see Jinek et al. (2012) Science 337:816; Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing and has been useful for zebrafish embryo genomic editing in vivo (see Hwang et al. (2013) Nat. Biotechnol., 31(3):227) with editing efficiencies similar to ZFNs and TALENs.

Accordingly in certain embodiments, a CRISPR/Cas endonuclease complex used in the constructs described herein (e.g., attached to an internalizing antibody) comprises a Cas protein and at least one to two ribonucleic acids (e.g., gRNAs) that are capable of directing the Cas protein to and hybridizing to a target motif of a target polynucleotide sequence. In some embodiments, a CRISPR/Cas endonuclease complex used in the constructs described herein comprises a Cas protein and one ribonucleic acid (e.g., gRNA) that us capable of directing the Cas protein to and hybridizing to a target motif of a target polynucleotide sequence.

As used herein, “protein” and “polypeptide” are used interchangeably to refer to a series of amino acid residues joined by peptide bonds (i.e., a polymer of amino acids) and include modified amino acids (e.g., phosphorylated, glycated, glycosolated, etc.) and amino acid analogs. Illustrative polypeptides or proteins include gene products, naturally occurring proteins, homologs, paralogs, fragments and other equivalents, variants, and analogs of the above.

In some embodiments, a Cas protein comprises a core Cas protein. Illustrative Cas core proteins include, but are not limited to, Cas1, Cas2, Cas3, Cas4, Cas5, Cash, Cas7, Cas8 and Cas9. In some embodiments, a Cas protein comprises a Cas protein of an E. coli subtype (also known as CASS2). Illustrative Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Illustrative Cas proteins of the Ypest subtype include, but are not limited to Csy1, Csy2, Csy3, and Csy4. In some embodiments, a Cas protein comprises a Cas protein of the Nmeni subtype (also known as CASS4). Illustrative Cas proteins of the Nmeni subtype include, but are not limited to Csn1 and Csn2. In some embodiments, a Cas protein comprises a Cas protein of the Dvulg subtype (also known as CASS1). Illustrative Cas proteins of the Dvulg subtype include Csd1, Csd2, and Cas5d. In some embodiments, a Cas protein comprises a Cas protein of the Tneap subtype (also known as CASS7). Illustrative Cas proteins of the Tneap subtype include, but are not limited to, Cst1, Cst2, Cas5t. In some embodiments, a Cas protein comprises a Cas protein of the Hmari subtype. Illustrative Cas proteins of the Hmari subtype include, but are not limited to Csh1, Csh2, and Cas5h. In some embodiments, a Cas protein comprises a Cas protein of the Apern subtype (also known as CASS5). Illustrative Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Illustrative Cas proteins of the Mtube subtype include, but are not limited to Csm1, Csm2, Csm3, Csm4, and Csm5. In some embodiments, a Cas protein comprises a RAMP module Cas protein. Illustrative RAMP module Cas proteins include, but are not limited to, Cmr1, Cmr2, Cmr3, Cmr4, Cmr5, and Cmr6.

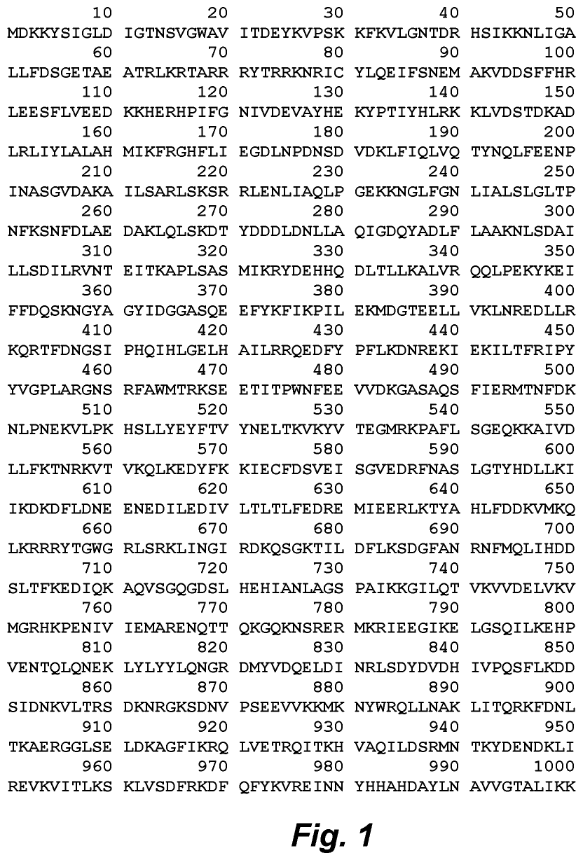

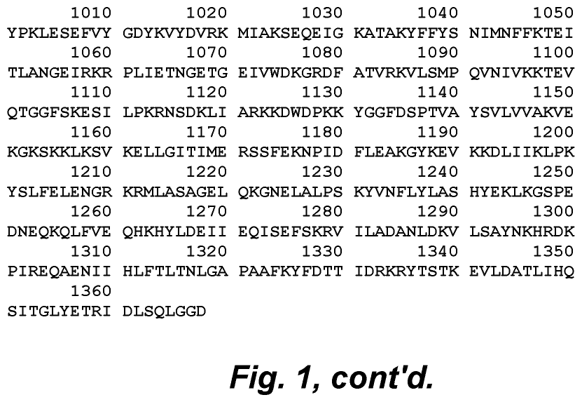

In some embodiments, the Cas protein is a Streptococcus pyogenes Cas9 protein (spCas9) or a functional portion thereof (see, e.g., FIG. 1, UniProtKB-Q99ZW2 (CAS9_STRP1)). In some embodiments, the Cas protein is a Staphylococcus aureus Cas9 protein (saCas9) or a functional portion thereof. In some embodiments, the Cas protein is a Streptococcus thermophilus Cas9 protein (stCas9) or a functional portion thereof. In some embodiments, the Cas protein is a Neisseria meningitides Cas9 protein (nmCas9) or a functional portion thereof. I n some embodiments, the Cas protein is a Treponema denticola Cas9 protein (tdCas9) or a functional portion thereof. In some embodiments, the Cas protein is Cas9 protein from any other bacterial species or functional portion thereof.

Type V and Type VI CRISPR/Cas Endonucleases

In certain embodiments compositions contemplated herein include, but are not limited to an antibody (e.g., an internalizing antibody) attached to a type V or type VI CRISPR/Cas endonuclease (e.g., the genome editing endonuclease is a type V or type VI CRISPR/Cas endonuclease) (e.g., Cpf1, C2c1, C2c2, C2c3). Type V and type VI CRISPR/Cas endonucleases are a type of class 2 CRISPR/Cas endonuclease. Examples of type V CRISPR/Cas endonucleases include but are not limited to: Cpf1, C2c1, and C2c3. An example of a type VI CRISPR/Cas endonuclease is C2c2. In some cases, a subject genome targeting composition includes a type V CRISPR/Cas endonuclease (e.g., Cpf1, C2c1, C2c3). In some cases, a Type V CRISPR/Cas endonuclease is a Cpf1 protein. In some cases, a subject genome targeting composition includes a type VI CRISPR/Cas endonuclease (e.g., C2c2)

Like type II CRISPR/Cas endonucleases, type V and VI CRISPR/Cas endonucleases form a complex with a corresponding guide RNA. The guide RNA provides target specificity to an endonuclease-guide RNA RNP complex by having a nucleotide sequence (a guide sequence) that is complementary to a sequence (the target site) of a target nucleic acid (as described elsewhere herein). The endonuclease of the complex provides the site-specific activity. In other words, the endonuclease is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the guide RNA.

Examples and guidance related to type V and type VI CRISPR/Cas proteins (e.g., cpf1, C2c1, C2c2, and C2c3 guide RNAs) can be found in the art, for example, see Zetsche et al. (2015) Cell, 163(3):759-771; Makarova et al. (2015) Nat. Rev. Microbiol. 13(11): 722-736; Shmakov et al. (2015) Mol. Cell, 60(3):385-397; and the like).

In some cases, the Type V or type VI CRISPR/Cas endonuclease (e.g., Cpf1, C2c1, C2c2, C2c3) is enzymatically active, e.g., the Type V or type VI CRISPR/Cas polypeptide, when bound to a guide RNA, cleaves a target nucleic acid. In some cases, the Type V or type VI CRISPR/Cas endonuclease (e.g., Cpf1, C2c1, C2c2, C2c3) exhibits reduced enzymatic activity relative to a corresponding wild-type a Type V or type VI CRISPR/Cas endonuclease (e.g., Cpf1, C2c1, C2c2, C2c3), and retains DNA binding activity.

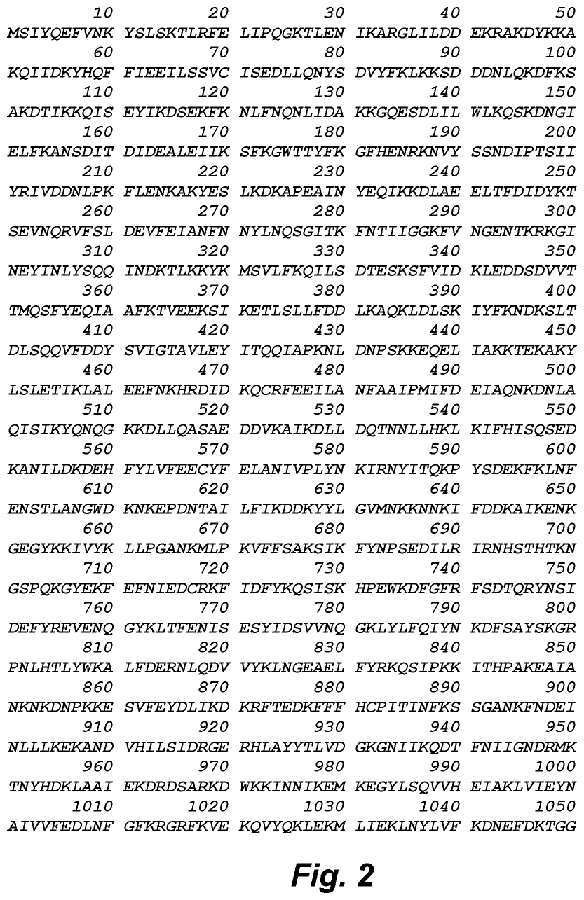

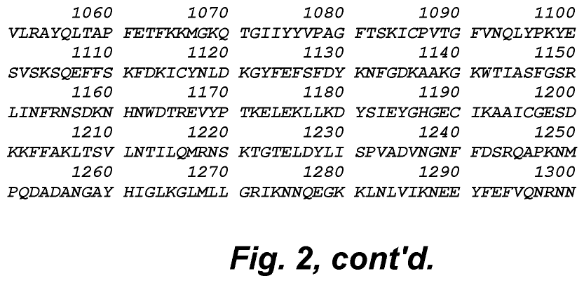

In some cases a type V CRISPR/Cas endonuclease is a Cpf1 protein or a functional portion thereof (see, e.g., FIG. 2, UniProtKB-AOQ7Q2 (CPF1_FRATN)). Cpf1 protein is a member of the type V CRISPR system and is a polypeptide comprising about 1300 amino acids. Cpf1 contains a RuvC-like endonuclease domain. Unlike Cas9, Cpf1 cleaves target DNA in a staggered pattern using a single ribonuclease domain. The staggered DNA double-stranded break results in a 4 or 5-nt 5′ overhang.

The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al. (2015) Genom. Bio. 16: 251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the .about.100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “Cas” includes both Cas9 and Cfp1 proteins. Accordingly, as used herein, a “CRISPR/Cas system” refers both CRISPR/Cas and/or CRISPR/Cfp1 systems, including both nuclease and/or transcription factor systems.

Accordingly, in certain embodiments the constructs described herein (e.g., an antibody attached to a Cas protein) the Cas protein is Cpf1 from any bacterial species or functional portion thereof. In some aspects, Cpf1 is a Francisella novicida U112 protein or a functional portion thereof. In some aspects, Cpf1 is a Acidaminococcus sp. BV3L6 protein or a functional portion thereof. In some aspects, Cpf1 is a Lachnospiraceae bacterium ND2006 protein or a function portion thereof.

In certain embodiments, Cas protein may be a “functional portion” or “functional derivative” of a naturally occurring Cas protein, or of a modified Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity (e.g., endonuclease activity) in common with a corresponding native sequence polypeptide. As used herein, “functional portion” refers to a portion of a Cas polypeptide that retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cpf1 protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cpf1 protein comprises a functional portion of a RuvC-like domain.

In certain embodiments a biological activity contemplated herein is the ability of the functional derivative to hydrolyze a DNA substrate into fragments. The term “derivative” encompasses both amino acid sequence variants of polypeptide, covalent modifications, and fusions thereof. In some aspects, a functional derivative may comprise a single biological property of a naturally occurring Cas protein. In other aspects, a function derivative may comprise a subset of biological properties of a naturally occurring Cas protein.

In view of the foregoing, the term “Cas polypeptide” as used herein encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically, recombinantly expressed, or by a combination of these procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In certain embodiments the Cas proteins used in the constructs described herein may be mutated to alter functionality. Illustrative selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in WO 02/077227.

In some embodiments, for example, the Cas9 protein is mutated in the HNH domain, rendering it unable to cleave the DNA strand that is complementary to the crRNA. In other illustrative, but non-limiting, embodiments, the Cas9 is mutated in the Rvu domain, making it incapable of cleaving the non-complimentary DNA strand. These mutations can result in the creation of Cas9 nickases. In some embodiments, two Cas nickases are used with two separate crRNAs to target a DNA, which results in two nicks in the target DNA at a specified distance apart. In other illustrative, but non-limiting, embodiments, both the HNH and Rvu endonuclease domains are altered to render a Cas9 protein that is unable to cleave a target DNA.

In certain embodiments the Cas proteins (e.g., Cas9 protein) comprise truncated Cas proteins. In one illustrative, but non-limiting, embodiment, the Cas9 comprises only the domain responsible for interaction with the crRNA or sgRNA and the target DNA.

In certain embodiments the Cas proteins comprising the constructs described herein comprise a Cas protein, or truncation thereof, fused to a different functional domain. In some aspects, the functional domain is an activation or a repression domain. In other aspects, the functional domain is a nuclease domain. In some embodiments, the nuclease domain is a FokI endonuclease domain (see, e.g. Tsai (2014) Nat. Biotechnol. doi:10.1038/nbt.2908). In some embodiments, the FokI domain comprises mutations in the dimerization domain.

The CRISPR/Cas system can also be used to inhibit gene expression. For example, Lei et al. (see, (2013) Cell, 152(5): 1173-1183) have shown that a catalytically dead Cas9 lacking endonuclease activity, when coexpressed with a guide RNA, generates a DNA recognition complex that can specifically interfere with transcriptional elongation, RNA polymerase binding, or transcription factor binding. This system, called CRISPR interference (CRISPRi), can efficiently repress expression of targeted genes. In certain embodiments the constructs described herein comprise an antibody (e.g., an internalizing antibody) attached to a CRISPRi complex.

### Mutant CRISPR/Cas Endonucleases.

A number of mutant endonucleases have been created that improve editing specificity and/or that improve editing efficiency. Such mutant endonucleases include, but are not limited to high fidelity (HiFi) Cas9 and the like.

One illustrative, but non-limiting embodiment, the mutant endonuclease comprises a Cas9 comprising a single point mutation, p.R691A (see, e.g., Vakulskas et al. (2018) Nat. Med., 24: 1216-1224

Another illustrative, but non-limiting mutant endonuclease comprise the Alt-R® S.p. HiFi Cas9 Nuclease from Integrated DNA Technologies (Skokie, Ill.).

In certain embodiments the CRISPR/Cas endonuclease or HiFi endonuclease is modified by the addition of 1, 2, 3, or 4, or more nuclear localization signals to enhance transport of the endonuclease to the cell nuclease. A nuclear localization signal or sequence (NLS) is an amino acid sequence that ‘tags’ a protein for import into the cell nucleus by nuclear transport. Typically, this signal consists of one or more short sequences of positively charged lysines or arginines exposed on the protein surface.

In certain embodiments NLSs can be further classified as either monopartite or bipartite. The major structural differences between the two is that the two basic amino acid clusters in bipartite NLSs are separated by a relatively short spacer sequence (hence bipartite—2 parts), while monopartite NLSs are not. The first NLS to be discovered was the sequence PKKKRKV (SEQ ID NO:29) in the SV40 Large T-antigen (a monopartite NLS) (Kalderon et al. (1984) Cell, 39(3 Pt 2): 499-509). The NLS of nucleoplasmin, KR[PAATKKAGQA]KKKK (SEQ ID NO:30), is the prototype of the ubiquitous bipartite signal: two clusters of basic amino acids, separated by a spacer of about 10 amino acids (Dingwall et al. (1988) J. Cell Biol. 107(3): 841-84). Both signals are recognized by importin α. Importin α contains a bipartite NLS itself, which is specifically recognized by importin β. The latter can be considered the actual import mediator.

One illustrative consensus sequence for a monopartite NLS is K-K/R-X-K/R (SEQ ID NO:31) where X is any amino acid (Dingwal et al. supra). Other NLSs include, but are not limited to the acidic M9 domain of hnRNP A1, the sequence KIPIK in yeast transcription repressor Matα2, and the complex signals of U snRNPs. Most of these NLSs appear to be recognized directly by specific receptors of the importin β family without the intervention of an importin α-like protein (see, e.g., Mattaj & Englmeier (1998) Annu. Rev. Biochem. 67(1): 265-306). A class of NLSs known as PY-NLSs has been proposed (see, Lee et al. (2006) Cell, 126 (3): 543-558. This PY-NLS motif, so named because of the proline-tyrosine amino acid pairing in it, allows the protein to bind to importin (32 (also known as transportin or karyopherin β2), which then translocates the cargo protein into the nucleus.

In certain embodiments the NLS(s) comprise a monopartite NLS including, but not limited to, the SV40 T antigen (PKKKRKV (SEQ ID NO:32)), the SV40 Vp3 (KKKRK (SEQ ID NO:33)), the Adenovirus Ela (KRPRP (SEQ ID NO:34)), the human c-myc (PAAKRVKLD (SEQ ID NO:35), RQRRNELKRSP (SEQ ID NO:36)), and derivatives thereof. In some embodiments, the peptide comprises a bipartite NLS including, but not limited to, nucleoplasmin (KRPAATKKAGQAKKKK (SEQ ID NO:37)), Xenopus N1 (VRKKRKTEEESPLKDKDAKKSKQE (SEQ ID NO:38)), mouse FGF3 (RLRRDAGGRGGVYEHLGGAPRRRK (SEQ ID NO:39)), PARP (KRKGDEVDGVDECAKKSKK (SEQ ID NO:40)), and derivatives thereof. In some embodiments, the NLS comprises a nonclassical NLS such as M9 peptide, NQSSNFGPMKGGNFGGRSSGPYGGGGQYFAKPRNQGGY (SEQ ID NO:41).

### Guide RNA (for CRISPR/Cas Endonucleases)

In various embodiments the constructs described herein comprise an antibody (e.g., an internalizing antibody) attached to a complex comprising a Cas protein and one or two RNAs (guide RNAs). In certain embodiments the complex comprise a Cas protein attached to a single guide RNA.

A nucleic acid molecule that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.”

In various embodiments the guide RNA provides target specificity to the complex (the RNP complex) by including a targeting segment, which includes a guide sequence (also referred to herein as a targeting sequence), which typically comprise a nucleotide sequence that is complementary to a sequence of a target nucleic acid

A guide RNA can be referred to by the protein to which it corresponds. For example, when the class 2 CRISPR/Cas endonuclease is a Cas9 protein, the corresponding guide RNA can be referred to as a “Cas9 guide RNA.” Likewise, as another example, when the class 2 CRISPR/Cas endonuclease is a Cpf1 protein, the corresponding guide RNA can be referred to as a “Cpf1 guide RNA.”

In some embodiments, a guide RNA includes two separate nucleic acid molecules (or two sequenced within a single molecule): an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

Cas9 Guide RNA

A nucleic acid molecule that binds to a Cas9 protein and targets the complex to a specific location within a target nucleic acid is referred to herein as a “Cas9 guide RNA.” In certain embodiments a Cas9 guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule.

In various embodiments the first segment (targeting segment) of a Cas9 guide RNA typically includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide. The protein-binding segment of a subject Cas9 guide RNA typically includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid

A Cas9 guide RNA and a Cas9 protein form a complex (e.g., bind via non-covalent interactions). The Cas9 guide RNA provides target specificity to the complex by including a targeting segment, which includes a guide sequence (a nucleotide sequence that is complementary to a sequence of a target nucleic acid). The Cas9 protein of the complex provides the site-specific activity (e.g., cleavage activity or an activity provided by the Cas9 protein when the Cas9 protein is a Cas9 fusion polypeptide, i.e., has a fusion partner). In other words, the Cas9 protein is guided to a target nucleic acid sequence (e.g., a target sequence in a chromosomal nucleic acid, e.g., a chromosome; a target sequence in an extrachromosomal nucleic acid, e.g., an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the Cas9 guide RNA.

The “guide sequence” also referred to as the “targeting sequence” of a Cas9 guide RNA can be modified so that the Cas9 guide RNA can target a Cas9 protein to any desired sequence of any desired target nucleic acid, with the exception that the protospacer adjacent motif (PAM) sequence can be taken into account. Thus, for example, a Cas9 guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some embodiments, a Cas9 guide RNA includes two separate nucleic acid molecules: an “activator” and a “targeter” and is referred to herein as a “dual Cas9 guide RNA”, a “double-molecule Cas9 guide RNA”, or a “two-molecule Cas9 guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

In various embodiments a Cas9 guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) typically comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator/tracrRNA) typically comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the Cas9 guide RNA. As such, each targeter molecule can be said to have a corresponding activator molecule (which has a region that hybridizes with the targeter). In various embodiments the targeter molecule additionally provides the targeting segment. Thus, in various embodiments, a targeter and an activator molecule (as a corresponding pair) can hybridize to form a Cas9 guide RNA. The exact sequence of a given crRNA or tracrRNA molecule is characteristic of the species in which the RNA molecules are found. A subject dual Cas9 guide RNA can include any corresponding activator and targeter pair.

The term “activator” or “activator RNA” is used herein to mean a tracrRNA-like molecule (tracrRNA: “trans-acting CRISPR RNA”) of a Cas9 dual guide RNA (and therefore of a Cas9 single guide RNA when the “activator” and the “targeter” are linked together by, e.g., intervening nucleotides). Thus, for example, a Cas9 guide RNA (dgRNA or sgRNA) typically comprises an activator sequence (e.g., a tracrRNA sequence). A tracr molecule (a tracrRNA) is a naturally existing molecule that hybridizes with a CRISPR RNA molecule (a crRNA) to form a Cas9 dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a Cas9 dual guide RNA (and therefore of a Cas9 single guide RNA when the “activator” and the “targeter” are linked together, e.g., by intervening nucleotides). Thus, for example, a Cas9 guide RNA (dgRNA or sgRNA) typically comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, in various embodiments, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

In various embodiments a Cas9 guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A Cas9 guide RNA (e.g., a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter typically comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator) typically comprises a stretch of nucleotides (a duplex-forming segment) that forms the other half of the dsRNA duplex of the protein-binding segment of the Cas9 guide RNA. In other words, a stretch of nucleotides of the targeter is complementary to and hybridizes with a stretch of nucleotides of the activator to form the dsRNA duplex of the protein-binding segment of a Cas9 guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a Cas9 guide RNA. The particular sequence of a given naturally existing crRNA or tracrRNA molecule is characteristic of the species in which the RNA molecules are found. Examples of suitable activator and targeter are well known in the art.

In various embodiments a Cas9 guide RNA (e.g., a dual guide RNA or a single guide RNA) can be comprised of any corresponding activator and targeter pair.

Targeting Segment of a Cas9 Guide RNA

The first segment of a subject guide nucleic acid typically includes a guide sequence (e.g., a targeting sequence)(a nucleotide sequence that is complementary to a sequence (a target site) in a target nucleic acid). In other words, the targeting segment of a subject guide nucleic acid can interact with a target nucleic acid (e.g., double stranded DNA (dsDNA)) in a sequence-specific manner via hybridization (i.e., base pairing). As such, the nucleotide sequence of the targeting segment may vary (depending on the target) and can determine the location within the target nucleic acid that the Cas9 guide RNA and the target nucleic acid will interact. The targeting segment of a Cas9 guide RNA can be modified (e.g., by genetic engineering)/designed to hybridize to any desired sequence (target site) within a target nucleic acid (e.g., a eukaryotic target nucleic acid such as genomic DNA).

In certain embodiments the targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

The nucleotide sequence (the targeting sequence) of the targeting segment that is complementary to a nucleotide sequence (target site) of the target nucleic acid can have a length of 10 nt or more. For example, the targeting sequence of the targeting segment that is complementary to a target site of the target nucleic acid can have a length of 12 nt or more, 15 nt or more, 18 nt or more, 19 nt or more, or 20 nt or more. In some cases, the nucleotide sequence (the targeting sequence) of the targeting segment that is complementary to a nucleotide sequence (target site) of the target nucleic acid has a length of 12 nt or more. In some cases, the nucleotide sequence (the targeting sequence) of the targeting segment that is complementary to a nucleotide sequence (target site) of the target nucleic acid has a length of 18 nt or more.

For example, in certain embodiments, the targeting sequence of the targeting segment that is complementary to a target sequence of the target nucleic acid can have a length of from 10 to 100 nucleotides (nt) (e.g., from 10 to 90 nt, from 10 to 75 nt, from 10 to 60 nt, from 10 to 50 nt, from 10 to 35 nt, from 10 to 30 nt, from 10 to 25 nt, from 10 to 22 nt, from 10 to 20 nt, from 12 to 100 nt, from 12 to 90 nt, from 12 to 75 nt, from 12 to 60 nt, from 12 to 50 nt, from 12 to 35 nt, from 12 to 30 nt, from 12 to 25 nt, from 12 to 22 nt, from 12 to 20 nt, from 15 to 100 nt, from 15 to 90 nt, from 15 to 75 nt, from 15 to 60 nt, from 15 to 50 nt, from 15 to 35 nt, from 15 to 30 nt, from 15 to 25 nt, from 15 to 22 nt, from 15 to 20 nt, from 17 to 100 nt, from 17 to 90 nt, from 17 to 75 nt, from 17 to 60 nt, from 17 to 50 nt, from 17 to 35 nt, from 17 to 30 nt, from 17 to 25 nt, from 17 to 22 nt, from 17 to 20 nt, from 18 to 100 nt, from 18 to 90 nt, from 18 to 75 nt, from 18 to 60 nt, from 18 to 50 nt, from 18 to 35 nt, from 18 to 30 nt, from 18 to 25 nt, from 18 to 22 nt, or from 18 to 20 nt). In some cases, the targeting sequence of the targeting segment that is complementary to a target sequence of the target nucleic acid has a length of from 15 nt to 30 nt. In some cases, the targeting sequence of the targeting segment that is complementary to a target sequence of the target nucleic acid has a length of from 15 nt to 25 nt. In some cases, the targeting sequence of the targeting segment that is complementary to a target sequence of the target nucleic acid has a length of from 18 nt to 30 nt. In some cases, the targeting sequence of the targeting segment that is complementary to a target sequence of the target nucleic acid has a length of from 18 nt to 25 nt. In some cases, the targeting sequence of the targeting segment that is complementary to a target sequence of the target nucleic acid has a length of from 18 nt to 22 nt. In some cases, the targeting sequence of the targeting segment that is complementary to a target site of the target nucleic acid is 20 nucleotides in length. In some cases, the targeting sequence of the targeting segment that is complementary to a target site of the target nucleic acid is 19 nucleotides in length.

In certain embodiments the percent complementarity between the targeting sequence (guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some cases, the percent complementarity between the targeting sequence of the targeting segment and the target site of the target nucleic acid is 100% over the seven contiguous 5′-most nucleotides of the target site of the target nucleic acid. In some cases, the percent complementarity between the targeting sequence of the targeting segment and the target site of the target nucleic acid is 60% or more over about 20 contiguous nucleotides. In some cases, the percent complementarity between the targeting sequence of the targeting segment and the target site of the target nucleic acid is 100% over the fourteen contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the seven contiguous 5′-most nucleotides of the target site of the target nucleic acid and as low as 0% or more over the remainder. In such a case, the targeting sequence can be considered to be 20 nucleotides in length.

In some cases, the percent complementarity between the targeting sequence of the targeting segment and the target site of the target nucleic acid is 100% over the 7 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 8 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 9 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 10 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 17 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 18 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over about 20 contiguous nucleotides.

In some cases, the percent complementarity between the targeting sequence of the targeting segment and the target site of the target nucleic acid is 100% over the 7 contiguous 5′-most nucleotides of the target site of the target nucleic acid and as low as 0% or more over the remainder. In such a case, the targeting sequence can be considered to be 7 nucleotides in length. In some cases, the percent complementarity between the targeting sequence of the targeting segment and the target site of the target nucleic acid is 100% over the 8 contiguous 5′-most nucleotides of the target site of the target nucleic acid and as low as 0% or more over the remainder. In such a case, the targeting sequence can be considered to be 8 nucleotides in length. In some cases, the percent complementarity between the targeting sequence of the targeting segment and the target site of the target nucleic acid is 100% over the 9 contiguous 5′-most nucleotides of the target site of the target nucleic acid and as low as 0% or more over the remainder. In such a case, the targeting sequence can be considered to be 9 nucleotides in length. In some cases, the percent complementarity between the targeting sequence of the targeting segment and the target site of the target nucleic acid is 100% over the 10 contiguous 5′-most nucleotides of the target site of the target nucleic acid and as low as 0% or more over the remainder. In such a case, the targeting sequence can be considered to be 10 nucleotides in length. In some cases, the percent complementarity between the targeting sequence of the targeting segment and the target site of the target nucleic acid is 100% over the 11 contiguous 5′-most nucleotides of the target site of the target nucleic acid and as low as 0% or more over the remainder. In such a case, the targeting sequence can be considered to be 11 nucleotides in length. In some cases, the percent complementarity between the targeting sequence of the targeting segment and the target site of the target nucleic acid is 100% over the 12 contiguous 5′-most nucleotides of the target site of the target nucleic acid and as low as 0% or more over the remainder. In such a case, the targeting sequence can be considered to be 12 nucleotides in length. In some cases, the percent complementarity between the targeting sequence of the targeting segment and the target site of the target nucleic acid is 100% over the 13 contiguous 5′-most nucleotides of the target site of the target nucleic acid and as low as 0% or more over the remainder. In such a case, the targeting sequence can be considered to be 13 nucleotides in length. In some cases, the percent complementarity between the targeting sequence of the targeting segment and the target site of the target nucleic acid is 100% over the 14 contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the 17 contiguous 5′-most nucleotides of the target site of the target nucleic acid and as low as 0% or more over the remainder. In such a case, the targeting sequence can be considered to be 17 nucleotides in length. In some cases, the percent complementarity between the targeting sequence of the targeting segment and the target site of the target nucleic acid is 100% over the 18 contiguous 5′-most nucleotides of the target site of the target nucleic acid and as low as 0% or more over the remainder. In such a case, the targeting sequence can be considered to be 18 nucleotides in length.

Protein-Binding Segment of a Cas9 Guide RNA

The protein-binding segment of a subject Cas9 guide RNA typically interacts with a Cas9 protein. The Cas9 guide RNA guides the bound Cas9 protein to a specific nucleotide sequence within target nucleic acid via the above mentioned targeting segment. The protein-binding segment of a Cas9 guide RNA typically comprises two stretches of nucleotides that are complementary to one another and hybridize to form a double stranded RNA duplex (dsRNA duplex). Thus, the protein-binding segment can include a dsRNA duplex. In some cases, the protein-binding segment also includes stem loop 1 (the “nexus”) of a Cas9 guide RNA. For example, in some cases, the activator of a Cas9 guide RNA (dgRNA or sgRNA) includes (i) a duplex forming segment that contributes to the dsRNA duplex of the protein-binding segment; and (ii) nucleotides 3′ of the duplex forming segment, e.g., that form stem loop 1 (the “nexus”). For example, in some cases, the protein-binding segment includes stem loop 1 (the “nexus”) of a Cas9 guide RNA. In some cases, the protein-binding segment includes 5 or more nucleotides (nt) (e.g., 6 or more, 7 or more, 8 or more, 9 or more, 10 or more, 11 or more, 12 or more, 15 or more, 20 or more, 30 or more, 40 or more, 50 or more, 60 or more, 70 or more, 75 or more, or 80 or more nt) 3′ of the dsRNA duplex (where 3′ is relative to the duplex-forming segment of the activator sequence).

The dsRNA duplex of the guide RNA (sgRNA or dgRNA) that forms between the activator and targeter is sometimes referred to herein as the “stem loop”. In addition, the activator (activator RNA, tracrRNA) of many naturally existing Cas9 guide RNAs (e.g., S. pygogenes guide RNAs) has 3 stem loops (3 hairpins) that are 3′ of the duplex-forming segment of the activator. The closest stem loop to the duplex-forming segment of the activator (3′ of the duplex forming segment) is called “stem loop 1” (and is also referred to herein as the “nexus”); the next stem loop is called “stem loop 2” (and is also referred to herein as the “hairpin 1”); and the next stem loop is called “stem loop 3” (and is also referred to herein as the “hairpin 2”).

In some cases, a Cas9 guide RNA (sgRNA or dgRNA) (e.g., a full length Cas9 guide RNA) has stem loops 1, 2, and 3. In some cases, an activator (of a Cas9 guide RNA) has stem loop 1, but does not have stem loop 2 and does not have stem loop 3. In some cases, an activator (of a Cas9 guide RNA) has stem loop 1 and stem loop 2, but does not have stem loop 3. In some cases, an activator (of a Cas9 guide RNA) has stem loops 1, 2, and 3.

In some cases, the activator (e.g., tracr sequence) of a Cas9 guide RNA (dgRNA or sgRNA) includes (i) a duplex forming segment that contributes to the dsRNA duplex of the protein-binding segment; and (ii) a stretch of nucleotides (e.g., referred to herein as a 3′ tail) 3′ of the duplex forming segment. In some cases, the additional nucleotides 3′ of the duplex forming segment form stem loop 1. In some cases, the activator (e.g., tracr sequence) of a Cas9 guide RNA (dgRNA or sgRNA) includes (i) a duplex forming segment that contributes to the dsRNA duplex of the protein-binding segment; and (ii) 5 or more nucleotides (e.g., 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, 50 or more, 60 or more, 70 or more, or 75 or more nucleotides) 3′ of the duplex forming segment. In some cases, the activator (activator RNA) of a Cas9 guide RNA (dgRNA or sgRNA) includes (i) a duplex forming segment that contributes to the dsRNA duplex of the protein-binding segment; and (ii) 5 or more nucleotides (e.g., 6 or more, 7 or more, 8 or more, 9 or more, 10 or more, 11 or more, 12 or more, 13 or more, 14 or more, 15 or more, 20 or more, 25 or more, 30 or more, 35 or more, 40 or more, 45 or more, 50 or more, 60 or more, 70 or more, or 75 or more nucleotides) 3′ of the duplex forming segment.

In some cases, the activator (e.g., tracr sequence) of a Cas9 guide RNA (dgRNA or sgRNA) includes (i) a duplex forming segment that contributes to the dsRNA duplex of the protein-binding segment; and (ii) a stretch of nucleotides (e.g., referred to herein as a 3′ tail) 3′ of the duplex forming segment. In some cases, the stretch of nucleotides 3′ of the duplex forming segment has a length in a range of from 5 to 200 nucleotides (nt) (e.g., from 5 to 150 nt, from 5 to 130 nt, from 5 to 120 nt, from 5 to 100 nt, from 5 to 80 nt, from 10 to 200 nt, from 10 to 150 nt, from 10 to 130 nt, from 10 to 120 nt, from 10 to 100 nt, from 10 to 80 nt, from 12 to 200 nt, from 12 to 150 nt, from 12 to 130 nt, from 12 to 120 nt, from 12 to 100 nt, from 12 to 80 nt, from 15 to 200 nt, from 15 to 150 nt, from 15 to 130 nt, from 15 to 120 nt, from 15 to 100 nt, from 15 to 80 nt, from 20 to 200 nt, from 20 to 150 nt, from 20 to 130 nt, from 20 to 120 nt, from 20 to 100 nt, from 20 to 80 nt, from 30 to 200 nt, from 30 to 150 nt, from 30 to 130 nt, from 30 to 120 nt, from 30 to 100 nt, or from 30 to 80 nt). In some cases, the nucleotides of the 3′ tail of an activator RNA are wild type sequences. It will be recognized that a number of different alternative sequences can be used.

Examples of various Cas9 proteins and Cas9 guide RNAs (as well as information regarding requirements related to protospacer adjacent motif (PAM) sequences present in targeted nucleic acids) can be found in the art (see, e.g., Jinek et al. (2012) Science, 337(6096): 816-821; Chylinski et al. (2013) RNA Biol. 10(5):726-737; Ma et al., (2013) Biomed. Res. Int. 2013: 270805; Hou et al. (2013) Proc. Natl. Acad. Sci. USA, 110(39): 15644-15649; Pattanayak et al. (2013) Nat. Biotechnol. 31(9): 839-843; Qi et al. (2013) Cell, 152(5): 1173-1183; Wang et al. (2013) Cell, 153(4): 910-918; Chen et. al. (2013) Nucl. Acids Res. 41(20): e19; Cheng et. al. (2012) Cell Res. 23(10): 1163-1171; Cho et. al. (2013) Genetics, 195(3): 1177-1180; DiCarlo et al. (2013) Nucl. Acids Res. 41(7): 4336-4343; Dickinson et. al. (2013) Nat. Meth. 10(10): 1028-1034; Ebina et. al. (2013) Sci. Rep. 3: 2510; Fujii et. al. (2013) Nucl. Acids Res. 41(20): e187; Hu et. al. (2013) Cell Res. 23(11): 1322-1325; Jiang et. al. (2013) Nucl. Acids Res. 41(20): e188; Larson et. al. (2013) Nat. Protoc. 8(11): 2180-2196; Mali et. at. (2013) Nat. Meth. 10(10): 957-963; Nakayama et. al. (2013) Genesis, 51(12): 835-843; Ran et. al. (2013) Nat. Protoc. 8(11): 2281-2308; Ran et. al. (2013) Cell 154(6): 1380-1389; Walsh et. al. (2013) Proc. Natl. Acad. Sci. USA, 110(39): 15514-15515; Yang et. al. (2013) Cell, 154(6): 1370-1379; Briner et al. (2014) Mol. Cell, 56(2): 333-339; and U.S. patents and patent Applications: U.S. Pat. Nos. 8,906,616; 8,895,308; 8,889,418; 8,889,356; 8,871,445; 8,865,406; 8,795,965; 8,771,945; 8,697,359; 2014/0068797; 2014/0170753; 2014/0179006; 2014/0179770; 2014/0186843; 2014/0186919; 2014/0186958; 2014/0189896; 2014/0227787; 2014/0234972; 2014/0242664; 2014/0242699; 2014/0242700; 2014/0242702; 2014/0248702; 2014/0256046; 2014/0273037; 2014/0273226; 2014/0273230; 2014/0273231; 2014/0273232; 2014/0273233; 2014/0273234; 2014/0273235; 2014/0287938; 2014/0295556; 2014/0295557; 2014/0298547; 2014/0304853; 2014/0309487; 2014/0310828; 2014/0310830; 2014/0315985; 2014/0335063; 2014/0335620; 2014/0342456; 2014/0342457; 2014/0342458; 2014/0349400; 2014/0349405; 2014/0356867; 2014/0356956; 2014/0356958; 2014/0356959; 2014/0357523; 2014/0357530; 2014/0364333; and 2014/0377868; all of which are incorporated herein by reference in their entirety.

In certain embodiments alternative PAM sequences may also be utilized, where a PAM sequence can be NAG as an alternative to NGG (Hsu (2014) supra.) using an S. pyogenes Cas9. Additional PAM sequences may also include those lacking the initial G (see, e.g., Sander and Joung (2014) Nature Biotech 32(4):347). In addition to the S. pyogenes encoded Cas9 PAM sequences, other PAM sequences can be used that are specific for Cas9 proteins from other bacterial sources. For example, the PAM sequences shown below in Table 3 (adapted from Sander and Joung, supra., and Esvelt et al. (2013) Nat. Meth. 10(11): 1116) are specific for these Cas9 proteins:

TABLE 3
Illustrative PAM sequences
from various species.

| | | SEQ |
|---|---|---|
| | | ID |
| Species | PAM | NO |
| S. pyogenes | NGG | |
| S. pyogenes | NAG | |
| S. mutans | NGG | |
| S. thermophilus | NGGNG | 42 |
| S. thermophilus | NNAAAW | 43 |
| S. thermophilus | NNAGAA | 44 |
| S. thermophilus | NNNGATT | 45 |
| C. jejuni | NNNNACA | 46 |
| N. meningitides | NNNNGATT | 47 |
| P. multocida | GNNNCNNA | 48 |
| F. novicida | NG | |

Thus, in certain embodiments, a suitable target sequence for use with a S. pyogenes CRISPR/Cas system can be chosen according to the following guideline: [n17, n18, n19, or n20](G/A)G (SEQ ID NO:49). Alternatively, in certain embodiments, the PAM sequence can follow the guideline G[n17, n18, n19, n20](G/A)G (SEQ ID NO:50). For Cas9 proteins derived from non-S. pyogenes bacteria, the same guidelines may be used where the alternate PAMs are substituted in for the S. pyogenes PAM sequences.

Guide RNAs Corresponding to Type V and Type VI CRISPR/Cas Endonucleases Cpf1 Guide RNA)

A guide RNA that binds to a type V or type VI CRISPR/Cas protein (e.g., Cpf1, C2c1, C2c2, C2c3), and targets the complex to a specific location within a target nucleic acid is referred to herein generally as a “type V or type VI CRISPR/Cas guide RNA”. An example of a more specific term is a “Cpf1 guide RNA.”

In various embodiments a type V or type VI CRISPR/Cas guide RNA (e.g., cpf1 guide RNA) can have a total length of from 30 nucleotides (nt) to 200 nt, e.g., from 30 nt to 180 nt, from 30 nt to 160 nt, from 30 nt to 150 nt, from 30 nt to 125 nt, from 30 nt to 100 nt, from 30 nt to 90 nt, from 30 nt to 80 nt, from 30 nt to 70 nt, from 30 nt to 60 nt, from 30 nt to 50 nt, from 50 nt to 200 nt, from 50 nt to 180 nt, from 50 nt to 160 nt, from 50 nt to 150 nt, from 50 nt to 125 nt, from 50 nt to 100 nt, from 50 nt to 90 nt, from 50 nt to 80 nt, from 50 nt to 70 nt, from 50 nt to 60 nt, from 70 nt to 200 nt, from 70 nt to 180 nt, from 70 nt to 160 nt, from 70 nt to 150 nt, from 70 nt to 125 nt, from 70 nt to 100 nt, from 70 nt to 90 nt, or from 70 nt to 80 nt). In some cases, a type V or type VI CRISPR/Cas guide RNA (e.g., cpf1 guide RNA) has a total length of at least 30 nt (e.g., at least 40 nt, at least 50 nt, at least 60 nt, at least 70 nt, at least 80 nt, at least 90 nt, at least 100 nt, or at least 120 nt).

In some cases, a Cpf1 guide RNA has a total length of 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt.

Like a Cas9 guide RNA, a type V or type VI CRISPR/Cas guide RNA (e.g., cpf1 guide RNA) can include a target nucleic acid-binding segment and a duplex-forming region (e.g., in some cases formed from two duplex-forming segments, i.e., two stretches of nucleotides that hybridize to one another to form a duplex)

In various embodiments the target nucleic acid-binding segment of a type V or type VI CRISPR/Cas guide RNA (e.g., cpf1 guide RNA) can have a length of from 15 nt to 30 nt, e.g., 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt. In some cases, the target nucleic acid-binding segment has a length of 23 nt. In some cases, the target nucleic acid-binding segment has a length of 24 nt. In some cases, the target nucleic acid-binding segment has a length of 25 nt.

In certain embodiments the guide sequence of a type V or type VI CRISPR/Cas guide RNA (e.g., cpf1 guide RNA) can have a length of from 15 nt to 30 nt (e.g., 15 to 25 nt, 15 to 24 nt, 15 to 23 nt, 15 to 22 nt, 15 to 21 nt, 15 to 20 nt, 15 to 19 nt, 15 to 18 nt, 17 to 30 nt, 17 to 25 nt, 17 to 24 nt, 17 to 23 nt, 17 to 22 nt, 17 to 21 nt, 17 to 20 nt, 17 to 19 nt, 17 to 18 nt, 18 to 30 nt, 18 to 25 nt, 18 to 24 nt, 18 to 23 nt, 18 to 22 nt, 18 to 21 nt, 18 to 20 nt, 18 to 19 nt, 19 to 30 nt, 19 to 25 nt, 19 to 24 nt, 19 to 23 nt, 19 to 22 nt, 19 to 21 nt, 19 to 20 nt, 20 to 30 nt, 20 to 25 nt, 20 to 24 nt, 20 to 23 nt, 20 to 22 nt, 20 to 21 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt. In some cases, the guide sequence has a length of 24 nt.

In certain embodiments the guide sequence of a type V or type VI CRISPR/Cas guide RNA (e.g., cpf1 guide RNA) can have 100% complementarity with a corresponding length of target nucleic acid sequence. The guide sequence can have less than 100% complementarity with a corresponding length of target nucleic acid sequence. For example, the guide sequence of a type V or type VI CRISPR/Cas guide RNA (e.g., cpf1 guide RNA) can have 1, 2, 3, 4, or 5 nucleotides that are not complementary to the target nucleic acid sequence. For example, in some cases, where a guide sequence has a length of 25 nucleotides, and the target nucleic acid sequence has a length of 25 nucleotides, in some cases, the target nucleic acid-binding segment has 100% complementarity to the target nucleic acid sequence. As another example, in some cases, where a guide sequence has a length of 25 nucleotides, and the target nucleic acid sequence has a length of 25 nucleotides, in some cases, the target nucleic acid-binding segment has 1 non-complementary nucleotide and 24 complementary nucleotides with the target nucleic acid sequence. As another example, in some cases, where a guide sequence has a length of 25 nucleotides, and the target nucleic acid sequence has a length of 25 nucleotides, in some cases, the target nucleic acid-binding segment has 2 non-complementary nucleotides and 23 complementary nucleotides with the target nucleic acid sequence.

In certain embodiments the duplex-forming segment of a type V or type VI CRISPR/Cas guide RNA (e.g., cpf1 guide RNA) (e.g., of a targeter RNA or an activator RNA) can have a length of from 15 nt to 25 nt (e.g., 15 nt, 16 nt, 17 nt, 18 nt, 19 nt, 20 nt, 21 nt, 22 nt, 23 nt, 24 nt, or 25 nt).

The RNA duplex of a type V or type VI CRISPR/Cas guide RNA (e.g., cpf1 guide RNA) can have a length of from 5 base pairs (bp) to 40 bp (e.g., from 5 to 35 bp, 5 to 30 bp, 5 to 25 bp, 5 to 20 bp, 5 to 15 bp, 5-12 bp, 5-10 bp, 5-8 bp, 6 to 40 bp, 6 to 35 bp, 6 to 30 bp, 6 to 25 bp, 6 to 20 bp, 6 to 15 bp, 6 to 12 bp, 6 to 10 bp, 6 to 8 bp, 7 to 40 bp, 7 to 35 bp, 7 to 30 bp, 7 to 25 bp, 7 to 20 bp, 7 to 15 bp, 7 to 12 bp, 7 to 10 bp, 8 to 40 bp, 8 to 35 bp, 8 to 30 bp, 8 to 25 bp, 8 to 20 bp, 8 to 15 bp, 8 to 12 bp, 8 to 10 bp, 9 to 40 bp, 9 to 35 bp, 9 to 30 bp, 9 to 25 bp, 9 to 20 bp, 9 to 15 bp, 9 to 12 bp, 9 to 10 bp, 10 to 40 bp, 10 to 35 bp, 10 to 30 bp, 10 to 25 bp, 10 to 20 bp, 10 to 15 bp, or 10 to 12 bp).

As an illustrative, but non-limiting example, a duplex-forming segment of a Cpf1 guide RNA can comprise a nucleotide sequence selected from (5′ to 3′): AAUUUCUACUGUUGUAGAU (SEQ ID NO:51), AAUUUCUGCUGUUGCAGAU (SEQ ID NO:52), AAUUUCCACUGUUGUGGAU (SEQ ID NO:53), AAUUCCUACUGUUGUAGGU (SEQ ID NO:54), AAUUUCUACUAUUGUAGAU (SEQ ID NO:55), AAUUUCUACUGCUGUAGAU (SEQ ID NO:56), AAUUUCUACUUUGUAGAU (SEQ ID NO:57), AAUUUCUACUUGUAGAU (SEQ ID NO:58), and the like. The guide sequence can then follow (5′ to 3′) the duplex forming segment.

A illustrative, but non-limiting example of an activator RNA (e.g., tracrRNA) of a C2c1 guide RNA (dual guide or single guide) is an RNA that includes the nucleotide sequence GAAUUUUUCAACGGGUGUGCCAAUGGCCACUUUCCAGGU GGCAAAGCCCGUUGAGCUUCUCAAAAAG (SEQ ID NO:59). In some illustrative, but non-limiting cases, a C2c1 guide RNA (dual guide or single guide) is an RNA that includes the nucleotide sequence GUCUAGAGGACAGAAUUUUUC AACGGGU GUGCCAAUGGCCA CUUUCCAGGUGGCAAAGCCCGUUGAGCUUCUCAAAAAG (SEQ ID NO:60). In some illustrative, but non-limiting cases, a C2c1 guide RNA (dual guide or single guide) is an RNA that includes the nucleotide sequence UCUAGAGGACAGAAUUUUUCAAC GGGUGUGCCA AUGGCCACU UUCCAGGUGGCAAAGCCCGUU GAGCUU CUCAAAAAG (SEQ ID NO:61). A non-limiting example of an activator RNA (e.g., tracrRNA) of a C2c1 guide RNA (dual guide or single guide) is an RNA that includes the nucleotide sequence ACUUUCCAGG CAAAGCCCGUUG AGCUUCUCAAAAAG (SEQ ID NO:62). In some illustrative, but non-limiting cases, a duplex forming segment of a C2c1 guide RNA (dual guide or single guide) of an activator RNA (e.g., tracrRNA) includes the nucleotide sequence AGCUUCUCA (SEQ ID NO:63) or the nucleotide sequence GCUUCUCA (SEQ ID NO:64) (the duplex forming segment from a naturally existing tracrRNA.

One illustrative but non-limiting example of a targeter RNA (e.g., crRNA) of a C2c1 guide RNA (dual guide or single guide) is an RNA with the nucleotide sequence CUGAGAAGUGGCAC (SEQ ID NO:65), where the Ns represent the guide sequence, that will vary depending on the target sequence, and although 20 Ns are depicted a range of different lengths are acceptable. In some cases, a duplex forming segment of a C2c1 guide RNA (dual guide or single guide) of a targeter RNA (e.g., crRNA) includes the nucleotide sequence CUGAGAAGUGGCAC (SEQ ID NO:66), or includes the nucleotide sequence CUGAGAAGU (SEQ ID NO:67), or includes the nucleotide sequence UGAGAAGUGGCAC (SEQ ID NO:68), or includes the nucleotide sequence UGAGAAGU (SEQ ID NO:69), and the like.

Examples and guidance related to type V or type VI CRISPR/Cas endonucleases and guide RNAs (as well as information regarding requirements related to protospacer adjacent motif (PAM) sequences present in targeted nucleic acids) can be found in the art (see, e.g., Zetsche et al. (2015) Cell, 163(3): 759-771; Makarova et al. (2015) Nat. Rev. Microbiol. 13(11): 722-736; Shmakov et al. (2015) Mol. Cell, 60(3): 385-397; and the like).

Modified Guide RNAs

It has been discovered that incorporation of bridged nucleic acids (BNAs) as well as locked nucleic acids (LNAs) at locations in CRISPR RNAs (crRNAs) broadly reduced off-target cleavage by the CRISPR endonuclease (e.g., Cas9). Accordingly, in certain embodiments, the guide RNAs incorporated into or used with the constructs described herein comprise one or more BNAs, and/or LNAs.

Locked Nucleic Acids.

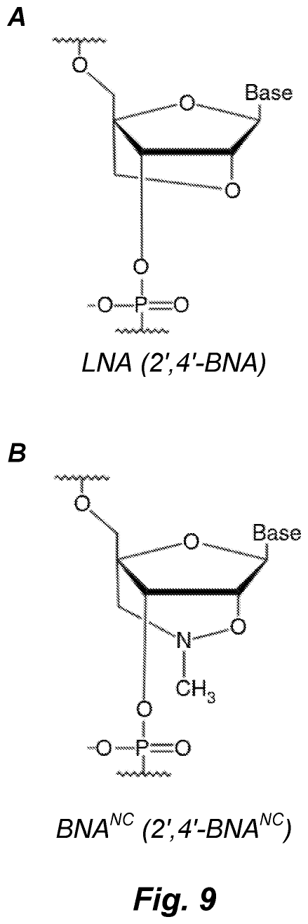

In certain embodiments the guide RNAs comprise one or more locked nucleic (LNAs) (see, e.g., FIG. 9, panel A). LNAs are conformationally restricted RNA nucleotides in which the 2′ oxygen in the ribose forms a covalent bond to the 4′ carbon, inducing N-type (C3′-endo) sugar puckering and a preference for an A-form helix (see, e.g., You, et al. (2006) Nucleic Acids Res. 34: e60). LNAs display improved base stacking and thermal stability compared to RNA, resulting in highly efficient binding to complementary nucleic acids and improved mismatch discrimination (see, e.g., You et al. (2006) Nucleic Acids Res. 34:e60; Vester & Wengel (2004) Biochem. 43: 13233-1324). They also display enhanced nuclease resistance (see, e.g., Vester & Wengel (2004) Biochem. 43: 13233-132429).

Accordingly, in various the guide RNAs described herein can comprise one or more LNAs. In certain embodiments the guide RNAs comprise, 1, 2, 3, 4, or more LNAs.

Bridged Nucleic Acids (BNAs).

Bridged nucleic acids (BNAs) are modified RNA nucleotides. They are sometimes also referred to as constrained or inaccessible RNA molecules. BNA monomers can contain a five-membered, six-membered or even a seven-membered bridged structure with a “fixed” C₃′-endo sugar puckering. The bridge is synthetically incorporated at the 2′, 4′-position of the ribose to afford a 2′, 4′-BNA monomer. The monomers can be incorporated into oligonucleotide polymeric structures using standard phosphoamidite chemistry. BNAs are structurally rigid oligo-nucleotides with increased binding affinities and stability.

It has been discovered that incorporation of bridged nucleic acids (BNAs) into CRSIPR guide RNAs can significantly improve CRISPR specificity. In particular, it has been demonstrated that N-methyl substituted BNAs (2′,4′-BNA^{NC}[N-Me]) (see, e.g., FIG. 9, panel B) when incorporated into Crispr crRNAs, can improve CRISPR accuracy by as much as 10,000 times (see, e.g., Cromwell et al. (2018) Nat. Comm. 9: 1448) have been shown to significantly improve Cas9 endonuclease specificity. Accordingly, in various the guide RNAs described herein can comprise one or more BNAs. In certain embodiments the guide RNAs comprise, 1, 2, 3, 4, or more BNA^{NC}s.

### Target Genomic DNA

The constructs described herein are effective to perform gene editing in a target genomic DNA. In certain embodiments the target genomic DNA is a DNA in a eukaryotic cell (e.g., a cell of a plant, an animal, a fungus, etc.). In certain embodiments the target DNA is a genomic DNA in a mammal (e.g., a human or non-human mammal). A target genomic DNA can be any genomic DNA in which the sequence is to be modified, e.g., by substitution and/or insertion and/or deletion of one or more nucleotides present in the target genomic DNA.

Target genes (target genomic DNA) include, but are not limited to, those genes involved in various diseases or conditions. In some cases, the target genomic DNA is mutated, such that it encodes a non-functional polypeptide, or such that a polypeptide encoded by the target genomic DNA is not synthesized in any detectable amount, or such that a polypeptide encoded by the target genomic DNA is synthesized in a lower than normal amount, such that an individual having the mutation has a disease. Such diseases include, but are not limited to, achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, Crigler-Najjer Syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), Glycogen Storage Disease Type IV, hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome. Other such diseases include, e.g., acquired immunodeficiencies, lysosomal storage diseases (e.g., Gaucher's disease, GM1, Fabry disease and Tay-Sachs disease), mucopolysaccahidosis (e.g., Hunter's disease, Hurler's disease), hemoglobinopathies (e.g., sickle cell diseases, HbC, α-thalassemia, β-thalassemia) and hemophilias.

For example, in some cases, the target genomic DNA comprises a mutation that gives rise to a trinucleotide repeat disease. Illustrative trinucleotide repeat diseases and target genes involved in trinucleotide repeat diseases Trinucleotide Repeat Diseases Gene DRPLA (Dentatorubropallidoluysian atrophy) ATN1 or DRPLA HD (Huntington's disease) HTT (Huntingtin) SBMA (Spinobulbar muscular atrophy or Androgen receptor on the Kennedy disease) X chromosome. SCA1 (Spinocerebellar ataxia Type 1) ATXN1 SCA2 (Spinocerebellar ataxia Type 2) ATXN2 SCA3 (Spinocerebellar ataxia Type 3 or ATXN3 Machado-Joseph disease) SCA6 (Spinocerebellar ataxia Type 6) CACNA1A SCAT (Spinocerebellar ataxia Type 7) ATXN7 SCA17 (Spinocerebellar ataxia Type 17) TBP FRAXA (Fragile X syndrome) FMR1, on the X-chromosome FXTAS (Fragile X-associated tremor/FMR1, on the X-ataxia syndrome) chromosome FRAXE (Fragile XE mental retardation) AFF2 or FMR2, on the X-chromosome FRDA (Friedreich's ataxia) FXN or X25, (frataxin-reduced expression) DM (Myotonic dystrophy) DMPK SCA8 (Spinocerebellar ataxia Type 8) OSCA or SCA8 SCA12 (Spinocerebellar ataxia Type 12) PPP2R2B or SCA12.

For example, in some cases, a suitable target genomic DNA is a β-globin gene, e.g., a β-globin gene with a sickle cell mutation. As another example, a suitable target genomic DNA is a Huntington's locus, e.g., an HTT gene, where the HTT gene comprises a mutation (e.g., a CAG repeat expansion comprising more than 35 CAG repeats) that gives rise to Huntington's disease. As another example, a suitable target genomic DNA is an adenosine deaminase gene that comprises a mutation that gives rise to severe combined immunodeficiency. As another example, a suitable target genomic DNA is a BCL11A gene comprising a mutation associated with control of the gamma-globin genes. As another example, a suitable target genomic DNA is a BCL11a enhancer.

Accordingly, in various embodiments the methods described herein involve the use of the constructs and/or pharmaceutical formulations described herein in the treatment of one or more of the above-identified pathologies.

### Donor Polynucleotide

In some cases, the compositions and methods described herein further comprise a donor template nucleic acid (“donor polynucleotide”). In some cases, a method described herein further comprises contacting the target DNA with a donor polynucleotide, where the donor polynucleotide, a portion of the donor polynucleotide, a copy of the donor polynucleotide, or a portion of a copy of the donor polynucleotide integrates into the target DNA (e.g., via homology-directed repair). In some cases, the method does not comprise contacting the cell with a donor polynucleotide (e.g., resulting in non-homologous end-joining). A donor poly nucleotide can be introduced into a target cell using any convenient technique for introducing nucleic acids into cells.

When it is desirable to insert a polynucleotide sequence into a target DNA sequence, a polynucleotide comprising a donor sequence to be inserted is provided to the cell (e.g., the target DNA is contacted with a donor polynucleotide in addition to a genome targeting composition (e.g., a genome editing endonuclease; or a genome-editing endonuclease and a guide RNA). By a “donor sequence” or “donor polynucleotide” it is meant a nucleic acid sequence to be inserted at the cleavage site induced by a genome-editing endonuclease. A suitable donor polynucleotide can be single stranded or double stranded. For example, in some cases, a donor polynucleotide is single stranded (e.g., in some cases can be referred to as an oligonucleotide), and in some cases a donor polynucleotide is double stranded (e.g., in some cases can be include two separate oligonucleotides that are hybridized). The donor polynucleotide will contain sufficient homology to a genomic sequence at the cleavage site, e.g., 70%, 80%, 85%, 90%, 95%, or 100% homology with the nucleotide sequences flanking the cleavage site, e.g., within 100 bases or less (e.g., 50 bases or less of the cleavage site, e.g., within 30 bases, within 15 bases, within 10 bases, within 5 bases, or immediately flanking the cleavage site), to support homology-directed repair between it and the genomic sequence to which it bears homology. Approximately 25 nucleotides (nt) or more (e.g., 30 nt or more, 40 nt or more, 50 nt or more, 60 nt or more, 70 nt or more, 80 nt or more, 90 nt or more, 100 nt or more, 150 nt or more, 200 nt or more, etc.) of sequence homology between a donor and a genomic sequence (or any integral value between 10 and 200 nucleotides, or more) can support homology-directed repair. For example, in some cases, the 5′ and/or the 3′ flanking homology arm (e.g., in some cases both of the flanking homology arms) of a donor polynucleotide can be 30 nucleotides (nt) or more in length (e.g., 40 nt or more, 50 nt or more, 60 nt or more, 70 nt or more, 80 nt or more, 90 nt or more, 100 nt or more, etc.). For example, in some cases, the 5′ and/or the 3′ flanking homology arm (e.g., in some cases both of the flanking homology arms) of a donor polynucleotide can have a length in a range of from 30 nt to 500 nt (e.g., 30 nt to 400 nt, 30 nt to 350 nt, 30 nt to 300 nt, 30 nt to 250 nt, 30 nt to 200 nt, 30 nt to 150 nt, 30 nt to 100 nt, 30 nt to 90 nt, 30 nt to 80 nt, 50 nt to 400 nt, 50 nt to 350 nt, 50 nt to 300 nt, 50 nt to 250 nt, 50 nt to 200 nt, 50 nt to 150 nt, 50 nt to 100 nt, 50 nt to 90 nt, 50 nt to 80 nt, 60 nt to 400 nt, 60 nt to 350 nt, 60 nt to 300 nt, 60 nt to 250 nt, 60 nt to 200 nt, 60 nt to 150 nt, 60 nt to 100 nt, 60 nt to 90 nt, 60 nt to 80 nt).

Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.”

In some cases, a donor polynucleotide is delivered to the cell (introduced into a cell) as part of viral vector (e.g., an adeno-associated virus (AAV) vector; a lentiviral vector; etc.). For example a viral DNA (e.g., AAV DNA) can include a donor polynucleotide sequence (donor sequence) (e.g., a virus, e.g., AAV, can include a DNA molecule that includes a donor polynucleotide sequence). In some cases, a donor polynucleotide is introduced into a cell as a virus (e.g., an AAV, e.g., the donor polynucleotide sequence is present as part of the viral DNA, e.g., AAV DNA) and the genome-editing endonuclease (e.g., ZFN; Cas9 protein; etc.) and, where applicable, a guide RNA are delivered by a different route. For example, in some cases, a donor polynucleotide is introduced into a cell as a virus (e.g., an AAV, e.g., the donor polynucleotide sequence is present as part of the viral DNA, e.g., AAV DNA) and a Cas9 protein and Cas9 guide RNA are delivered as part of a separate expression vector. In some cases, a donor polynucleotide is introduced into a cell as a virus (e.g., an AAV, e.g., the donor polynucleotide sequence is present as part of the viral DNA, e.g., AAV DNA) and a Cas9 protein and Cas9 guide RNA are delivered as part of a ribonucleoprotein complex (RNP). In some cases: (i) a donor polynucleotide is introduced into a cell as a virus (e.g., an AAV, e.g., the donor polynucleotide sequence is present as part of the viral DNA, e.g., AAV DNA), (ii) a Cas9 guide RNA is delivered as either an RNA or DNA encoding the RNA, and (iii) a Cas9 protein is delivered as a protein or as a nucleic acid encoding the protein (e.g., RNA or DNA).

In some cases, a recombinant viral vector (e.g., a recombinant AAV vector) comprising a donor polynucleotide is introduced into a cell before a Cas9-guide RNA RNP is introduced into the cell. For example, in some cases, a recombinant viral vector (e.g., a recombinant AAV vector) comprising a donor polynucleotide is introduced into a cell from 2 hours to 72 hours (e.g., from 2 hours to 4 hours, from 4 hours to 8 hours, from 8 hours to 12 hours, from 12 hours to 24 hours, from 24 hours to 48 hours, or from 48 hours to 72 hours) before the Cas9-guide RNA RNP is introduced into the cell.

### Methods of Use.

In certain embodiments methods of performing gene editing on a cell are provided, where the method involves contacting the cell with a construct (e.g., a targeting moiety-directed Cas endonuclease/guide RNA complex). The targeting moiety (e.g., antibody) directs the construct the target cell and/or mediates uptake of the construct by the cell. The guide RNA in the complex, typically guides the Cas endonuclease to a specific location in the genome of said cell.

In certain embodiments the method is performed on a cell ex vivo. In certain embodiments the method involves an autologous cell transfer. Thus, for example, the cell is derived from a subject to be treated, the genome of the cell is modified using a construct described herein, and the cell is transferred back into the subject.

In various illustrative, but non-limiting methods, the cell can be any eukaryotic cell(s), for example a plant cell or a mammalian cell or cell line, including, but not limited to COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodopterafugiperda (Sf), or fungal cells such as Saccharomyces, Pichia and Schizosaccharomyces. In certain embodiments, the cell line is a CHO, MDCK or HEK293 cell line.

Primary cells can also be edited as described herein. Such cells include, but are not limited to fibroblasts, blood cells (e.g., red blood cells, white blood cells), liver cells, kidney cells, neural cells, and the like. Suitable cells also include stem cells such as, by way of example, embryonic stem cells, induced pluripotent stem cells (iPSCs), hematopoietic stem cells, neuronal stem cells and mesenchymal stem cells.

As explained above, however, the constructs described herein are effective to perform gene editing in situ, e.g., directly in a subject to be treated. Thus, in certain embodiments, the cell that is to be edited is a cell in vivo in a subject and the contacting comprises administering the construct (or a pharmaceutical formulation comprising the construct) to the subject. In certain embodiments the methods involve administering the construct via a route selected from the group consisting of intraperitoneal administration, topical administration, oral administration, inhalation administration, transdermal administration, subdermal depot administration, and rectal administration. In certain embodiments the subject is a human, while in other embodiments, the subject is a non-human mammal.

In certain embodiments the methods involve treating a subject in need of such treatment. In certain embodiments the subjects are subjects with a pathology selected from the group consisting of achondroplasia, achromatopsia, acid maltase deficiency, adenosine deaminase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, Crigler-Najjer Syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), Glycogen Storage Disease Type IV, hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, and X-linked lymphoproliferative syndrome. Other such diseases include, e.g., acquired immunodeficiencies, lysosomal storage diseases (e.g., Gaucher's disease, GM1, Fabry disease and Tay-Sachs disease), mucopolysaccahidosis (e.g., Hunter's disease, Hurler's disease), hemoglobinopathies (e.g., sickle cell diseases, HbC, α-thalassemia, β-thalassemia), and hemophilias.

### Pharmaceutical Formulations.

In certain embodiments, the targeted-Cas endonuclease/guide RNA complexes described herein are administered to a mammal to edit one or more regions of the genome in one or more cells or tissues. In certain embodiments the constructs are used to treat a pathology that can be treated/corrected by such editing of the genome.

The targeted-Cas endonuclease/guide RNA complexes described herein can be administered in the “native” form or, if desired, in the form of salts, esters, amides, derivatives, and the like, provided the salt, ester, amide, or derivative is suitable pharmacologically, e.g., effective in the present method(s). Salts, esters, amides, and other derivatives of the targeted-Cas endonuclease/guide RNA complexes described herein can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, a pharmaceutically acceptable salt can be prepared for any compound described herein having a functionality capable of forming a salt (e.g., such as a carboxylic acid functionality of the compounds described herein). A pharmaceutically acceptable salt is any salt that retains the activity of the parent compound and does not impart any deleterious or untoward effect on the subject to which it is administered and in the context in which it is administered.

Methods of pharmaceutically formulating the targeted-Cas endonuclease/guide RNA complexes described herein as salts, esters, amides, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the compounds described herein can include halide salts, such as may be prepared using hydrochloric or hydrobromic acids. Conversely, preparation of basic salts of the targeted-Cas endonuclease/guide RNA complexes described herein can be prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (e.g., break down into the individual entities of drug and counterion) in an aqueous environment.

In various embodiments, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, formate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent (e.g., targeted-Cas endonuclease/guide RNA complexes described herein). In certain embodiments, the esters are typically acyl-substituted derivatives of free alcohol groups, e.g., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the compounds identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., amyloidogenic pathologies).

The active agent(s) described herein (e.g., targeted-Cas endonuclease/guide RNA complexes) can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the targeted-Cas endonuclease/guide RNA complexes. Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the targeted-Cas endonuclease/guide RNA complexes, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g., calcium carbonate, carboxymethylcellulose calcium, sodium starch glycollate, crospovidone etc.), a binder (e.g., alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., targeted-Cas endonuclease/guide RNA complexes) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the targeted-Cas endonuclease/guide RNA complexes described herein and on the particular physio-chemical characteristics of the targeted-Cas endonuclease/guide RNA complexes.

In certain embodiments, the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectable, implantable sustained-release formulations, mucoadherent films, topical varnishes, lipid complexes, etc.

Pharmaceutical compositions comprising the targeted-Cas endonuclease/guide RNA complexes described herein can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the targeted-Cas endonuclease/guide RNA complexes described herein into preparations that can be used pharmaceutically. Proper formulation is dependent upon the route of administration chosen.

Systemic formulations include, but are not limited to, those designed for administration by injection, e.g., subcutaneous, intravenous, intramuscular, intrathecal or intraperitoneal injection, as well as those designed for transdermal, transmucosal oral or pulmonary administration. For injection, the targeted-Cas endonuclease/guide RNA complexes described herein can be formulated in aqueous solutions, preferably in physiologically compatible buffers such as Hanks solution, Ringer's solution, or physiological saline buffer and/or in certain emulsion formulations. The solution can contain formulatory agents such as suspending, stabilizing and/or dispersing agents. In certain embodiments, targeted-Cas endonuclease/guide RNA complexes described herein can be provided in powder form for constitution with a suitable vehicle, e.g., sterile pyrogen-free water, before use. For transmucosal administration, penetrants appropriate to the barrier to be permeated can be used in the formulation. Such penetrants are generally known in the art.

For oral administration, the compounds can be readily formulated by combining the targeted-Cas endonuclease/guide RNA complexes with pharmaceutically acceptable carriers well known in the art. Such carriers enable the compounds described herein to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the targeted-Cas endonuclease/guide RNA complexes described herein are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In various embodiments, the targeted-Cas endonuclease/guide RNA complexes described herein can be formulated in rectal or vaginal compositions such as suppositories or retention enemas, e.g., containing conventional suppository bases such as cocoa butter or other glycerides.

In addition to the formulations described previously, targeted-Cas endonuclease/guide RNA complexes described herein may also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to protect and deliver pharmaceutically active compounds. Certain organic solvents such as dimethylsulfoxide also can be employed, although usually at the cost of greater toxicity. Additionally, the compounds may be delivered using a sustained-release system, such as semipermeable matrices of solid polymers containing the therapeutic agent. Various uses of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

In certain embodiments, the targeted-Cas endonuclease/guide RNA complexes and/or formulations described herein are administered orally. This is readily accomplished by the use of tablets, caplets, lozenges, liquids, and the like.

In certain embodiments, the targeted-Cas endonuclease/guide RNA complexes described herein are administered systemically (e.g., orally, or as an injectable) in accordance with standard methods well known to those of skill in the art. In other embodiments, the agents can also be delivered through the skin using conventional transdermal drug delivery systems, e.g., transdermal “patches” wherein the compound(s) and/or formulations described herein are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to targeted-Cas endonuclease/guide RNA complexes and any other materials that are present.

In certain embodiments, one or more targeted-Cas endonuclease/guide RNA complexes described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In certain embodiments, the targeted-Cas endonuclease/guide RNA complexes described herein are suitable for oral administration. In various embodiments, the compound(s) in the oral compositions can be either coated or non-coated. The preparation of enteric-coated particles is disclosed for example in U.S. Pat. Nos. 4,786,505 and 4,853,230.

In various embodiments, compositions contemplated herein typically comprise one or more of the various targeted-Cas endonuclease/guide RNA complexes described herein in an effective amount to achieve a pharmacological effect or therapeutic improvement without undue adverse side effects. Illustrative pharmacological effects or therapeutic improvements include, but are not limited to a reduction or cessation in one or more symptoms of the pathology being treated.

In various embodiments, the typical dose of targeted-Cas endonuclease/guide RNA complexes described herein varies and will depend on various factors such as the individual requirements of the patients and the disease to be diagnosed and/or treated. In general, the daily dose of compounds can be in the range of 1-1,000 mg or 1-800 mg, or 1-600 mg, or 1-500 mg, or 1-400 mg. In one illustrative embodiment a standard approximate amount of targeted-Cas endonuclease/guide RNA complexes described above present in the composition can be typically about 1 to 1,000 mg, more preferably about 5 to 500 mg, and most preferably about 10 to 100 mg. In certain embodiments the targeted-Cas endonuclease/guide RNA complexes described herein are administered only once, or for follow-up as required. In certain embodiments the targeted-Cas endonuclease/guide RNA complexes described herein once a day for a certain period of time, in certain embodiments, administered twice a day, in certain embodiments, administered 3 times/day, and in certain embodiments, administered 4, or 6, or 6 or 7, or 8 times/day.

In certain embodiments the active ingredients (targeted-Cas endonuclease/guide RNA complexes described herein) are formulated in a single oral dosage form containing all active ingredients. Such oral formulations include solid and liquid forms. It is noted that solid formulations typically provide improved stability as compared to liquid formulations and can often afford better patient compliance.

In one illustrative embodiment, the one or more of the targeted-Cas endonuclease/guide RNA complexes described herein are formulated in a single solid dosage form such as single- or multi-layered tablets, suspension tablets, effervescent tablets, powder, pellets, granules or capsules comprising multiple beads as well as a capsule within a capsule or a double chambered capsule. In another embodiment, the targeted-Cas endonuclease/guide RNA complexes described herein may be formulated in a single liquid dosage form such as suspension containing all active ingredients or dry suspension to be reconstituted prior to use.

In certain embodiments, the targeted-Cas endonuclease/guide RNA complexes described herein are formulated as enteric-coated delayed-release granules or as granules coated with non-enteric time-dependent release polymers in order to avoid contact with the gastric juice. Non-limiting examples of suitable pH-dependent enteric-coated polymers are: cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. A suitable commercially available enteric material, for example, is sold under the trademark EUDRAGIT L 100-55®. This coating can be spray coated onto a substrate.

Illustrative non-enteric-coated time-dependent release polymers include, for example, one or more polymers that swell in the stomach via the absorption of water from the gastric fluid, thereby increasing the size of the particles to create thick coating layer. The time-dependent release coating generally possesses erosion and/or diffusion properties that are independent of the pH of the external aqueous medium. Thus, the active ingredient is slowly released from the particles by diffusion or following slow erosion of the particles in the stomach.

Illustrative non-enteric time-dependent release coatings are for example: film-forming compounds such as cellulosic derivatives, such as methylcellulose, hydroxypropyl methylcellulose (HPMC), hydroxyethylcellulose, and/or acrylic polymers including the non-enteric forms of the EUDRAGIT® brand polymers. Other film-forming materials can be used alone or in combination with each other or with the ones listed above. These other film forming materials generally include, for example, poly(vinylpyrrolidone), Zein, poly(ethylene glycol), poly(ethylene oxide), poly(vinyl alcohol), poly(vinyl acetate), and ethyl cellulose, as well as other pharmaceutically acceptable hydrophilic and hydrophobic film-forming materials. These film-forming materials may be applied to the substrate cores using water as the vehicle or, alternatively, a solvent system. Hydro-alcoholic systems may also be employed to serve as a vehicle for film formation.

Other materials suitable for making the time-dependent release coating of the compounds described herein include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include, but are not limited to poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

While the targeted-Cas endonuclease/guide RNA complexes and methods of use thereof are described herein with respect to use in humans, they are also suitable for animal, e.g., veterinary use. Thus certain illustrative organisms include, but are not limited to humans, non-human primates, canines, equines, felines, porcines, ungulates, lagomorphs, and the like.

The foregoing formulations and administration methods are intended to be illustrative and not limiting. It will be appreciated that, using the teaching provided herein, other suitable formulations and modes of administration can be readily devised.

### Kits.

In various embodiments the agents described herein (targeted-Cas endonuclease/guide RNA complexes described herein) can be provided in kits. In certain embodiments the kits comprise the targeted-Cas endonuclease/guide RNA complexes described herein enclosed in multiple or single dose containers. In certain embodiments the kits can comprises component parts that can be assembled for use. For example, an targeted-Cas endonuclease/guide RNA complexes in lyophilized form and a suitable diluent may be provided as separated components for combination prior to use. In certain embodiments a kit may include targeted-Cas endonuclease/guide RNA complexes described herein and a second therapeutic agent for co-administration. The active agent and second therapeutic agent may be provided as separate component parts. A kit may include a plurality of containers, each container holding one or more unit dose of the targeted-Cas endonuclease/guide RNA complexes. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration, e.g., as described herein.

In certain embodiments the kits can further comprise instructional/informational materials. In certain embodiments the informational material(s) indicate that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. In certain embodiments the informational material(s) may indicate that anaphylaxis can be fatal and may occur when any foreign substance is introduced into the body. In certain embodiments the informational material may indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

While the instructional materials typically comprise written or printed materials they are not limited to such. Any medium capable of storing such instructions and communicating them to an end user is contemplated herein. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments, the kits can comprise one or more packaging materials such as, for example, a box, bottle, tube, vial, container, sprayer, insufflator, intravenous (I.V.) bag, envelope, and the like, and at least one unit dosage form of an agent comprising active agent(s) described herein and a packaging material. In some embodiments, the kits also include instructions for using the composition as prophylactic, therapeutic, or ameliorative treatment for the disease of concern.

In some embodiments, the articles of manufacture can comprise one or more packaging materials such as, for example, a box, bottle, tube, vial, container, sprayer, insufflator, intravenous (I.V.) bag, envelope, and the like; and a first composition comprising at least one unit dosage form of an agent comprising one or more allosteric BACE inhibitor(s) described herein within the packaging material.

### EXAMPLES

The following examples are offered to illustrate, but not to limit the claimed invention.

### Example 1

### Development of In Vivo-Compatible Cell-Specific Targeting and Editing of T Cells

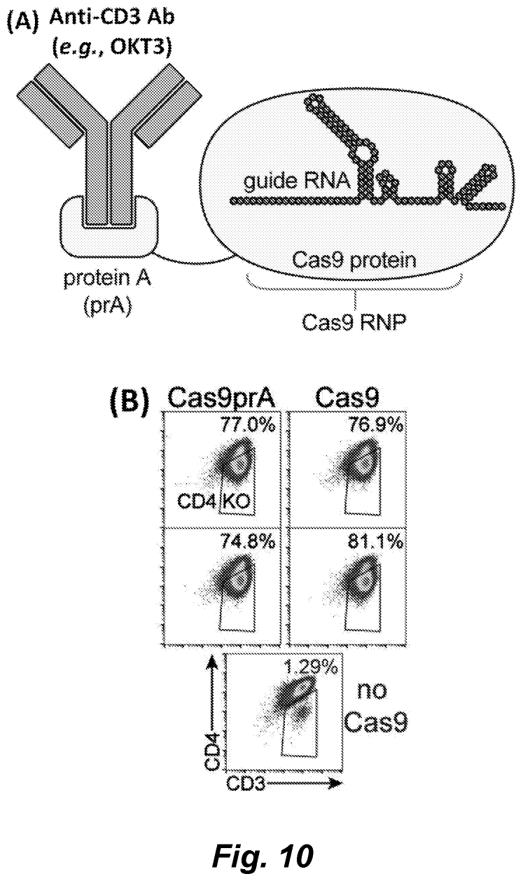

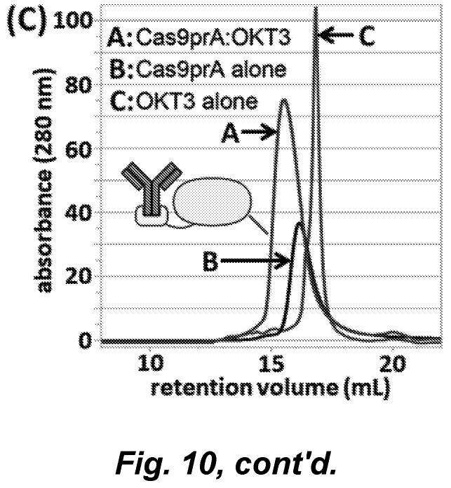

In this example, we demonstrate cell-specific targeting of T cells by a Cas9 RNP that is tethered to an antibody against CD3, a T cell specific marker (see, e.g., FIG. 10, panel A). This targeting will induce endocytosis of the Cas9 RNP that is capable of specific binding and uptake into T cells with subsequent efficient genome editing.

Preliminary Studies

Molecular Targeting of T Cells.

Antibodies (Ab) represent a reliable and well-characterized means of selectively targeting a given cell type. To allow recruitment of an Ab to Cas9, we purified a fusion construct, Cas9-prA, bearing a fragment of protein A that can bind the constant region (F_{c}) of an Ab with high affinity (Sjodahl (1977) Eur. J. Biochem. 73(2): 343-351; Choe et al. (2016) Materials 9(12): 994). In this illustrative, but non-limiting, embodiment, we chose this approach to ensure a 1:1 association between Cas9:Ab to keep the complex as small as possible. Cas9-prA was observed to be fully active via nucleofection e.g., electroporation) into primary T cells (FIG. 10, panel B). To facilitate flow cytometry experiments, Cas9 and Cas9prA were chemically labeled with Alexa Fluor 488 as described previously (Rouet et al. (2018) J. Am. Chem. Soc. 140(21): 6596-6603). After confirming complex formation between Cas9prA and a heterogeneous IgG population via size-exclusion chromatography, the same verification was performed with OKT3 (used therapeutically as muromonab), a well-characterized anti-CD3 Ab (FIG. 10, panel C) (Kung et al. (1979) Science, 206(4416): 347-349; Kuhn & Weiner (2016) Immunotherapy, 8(8): 889-906). Thus we established two fluorescent complexes, Cas9prA:OKT3 and Cas9prA:IgG to test for the ability of OKT3 to direct molecular targeting of Cas9 RNP to T cells.

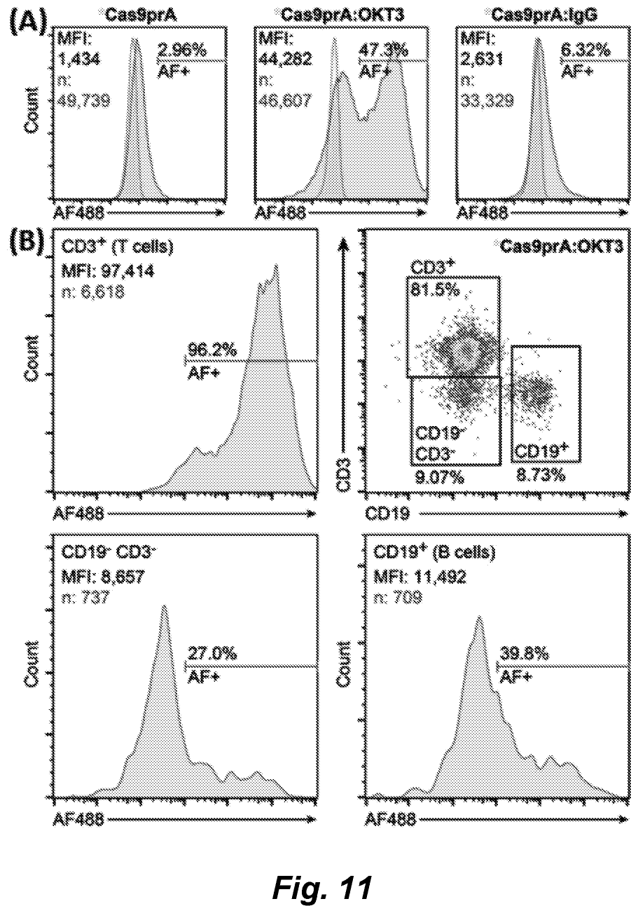

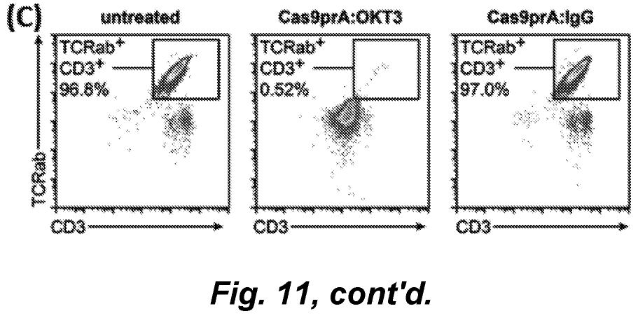

The Cas9prA:OKT3 complex, bound to guide RNA (gRNA) to form RNP, was tested for its ability to associate with (e.g., bind to and/or be taken up by) T cells specifically, as compared to Cas9prA RNP complexed with heterogeneous IgG (a negative control). The OKT3 directed fluorescently-labeled Cas9prA to bind the T cells with much greater frequency than when Cas9prA was alone or bound to heterogeneous IgG (FIG. 11, panel A).

A similar experiment was performed in peripheral blood mononuclear cells (PBMCs), and Cas9prA:OKT3 RNP was observed binding to T cells preferentially after 30 minutes, with only background levels of binding to B cells (FIG. 11, panel B). T cells co-localizing with Cas9prA:OKT3 (as detected by fluorescence) were found to have drastically diminished levels of surface TCR at 30 minutes, suggesting likely internalization of the Cas9prA:OKT3 complex (FIG. 11, panel C). This would not be unexpected, since OKT3 can trigger internalization of the TCR upon binding to CD3 (Kuhn & Weiner (2016) Immunotherapy, 8(8): 889-906).

To our knowledge, this is the first demonstration of molecular targeting being used to preferentially associate a genome editing enzyme with a particular cell type in a mixed population of cells. The non-covalent, yet high affinity association between Cas9prA and the Ab of our choosing means the complex is formed simply by mixing the two components, providing exceptional versatility as the platform is optimized.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A construct for performing gene editing in a mammalian cell, said construct comprising:
a targeting moiety that binds a cell surface marker, where said targeting moiety is attached to a ribonucleoprotein complex comprising a class 2 CRISPR/Cas endonuclease complexed with a corresponding CRISPR/Cas guide RNA that hybridizes to a target sequence within the genomic DNA of the cell.

2. The construct of claim 1, wherein said targeting moiety is selected from the group consisting of an antibody, a DNA/RNA or peptide aptamer, an anticalin, a lectin, and a DARPIN.

3. The construct of claim 1, wherein said class 2 CRISPR/Cas endonuclease is a type II CRISPR/Cas endonuclease.

4. The construct of claim 3, wherein:
the class 2 CRISPR/Cas endonuclease is a Cas9 polypeptide and the corresponding CRISPR/Cas guide RNA is a Cas9 guide RNA; or
the class 2 CRISPR/Cas endonuclease is a type V or type VI CRISPR/Cas endonuclease.

5. The construct of claim 4, wherein:
said Cas9 protein is selected from the group consisting of a Streptococcus pyogenes Cas9 protein (spCas9) or a functional portion thereof, a Staphylococcus aureus Cas9 protein (saCas9) or a functional portion thereof, a Streptococcus thermophilus Cas9 protein (stCas9) or a functional portion thereof, a Neisseria meningitides Cas9 protein (nmCas9) or a functional portion thereof, and a Treponema denticola Cas9 protein (tdCas9) or a functional portion thereof; or
the class 2 CRISPR/Cas polypeptide is selected from the group consisting of a Cpf1 polypeptide or a functional portion thereof, a C2c1 polypeptide or a functional portion thereof, a C2c3 polypeptide or a functional portion thereof, and a C2c2 polypeptide or a functional portion thereof; or
the class 2 CRISPR/Cas endonuclease is a the class 2 CRISPR/Cas endonuclease is a high fidelity (HiFi) mutant Cas9 polypeptide and the corresponding CRISPR/Cas guide RNA is a Cas9 guide RNA; or
the class 2 CRISPR/Cas endonuclease is a the class 2 CRISPR/Cas endonuclease is a high fidelity (HiFi) mutant Cas9 polypeptide and the corresponding CRISPR/Cas guide RNA is a Cas9 guide RNA and said mutant cas9 comprises an Alt-R® CRISPR-Cas9 or an R691A Cas9 mutant.

6-13. (canceled)

14. The construct of claim 5, wherein:
said mutant cas9 comprises a Cas9 enhanced with one, two, or three nuclear localization signals (NLS); or
said mutant cas9 comprises a Cas9 enhanced with one, two, or three nuclear localization signals (NLS) where said NLS comprise an NLS selected from the group consisting of the SV40 T antigen (PKKKRKV (SEQ ID NO:32)), the SV40 Vp3 (KKKRK (SEQ ID NO:33)), the Adenovirus Ela (KRPRP (SEQ ID NO:34)), the human c-myc (PAAKRVKLD (SEQ ID NO:35), RQRRNELKRSP (SEQ ID NO:36)), nucleoplasmin (KRPAATKKAGQAKKKK (SEQ ID NO:37)), Xenopus N1 (VRKKRKTEEESPLKDKDAKKSKQE (SEQ ID NO:38)), mouse FGF3 (RLRRDAGGRGGVYEHLGGAPRRRK (SEQ ID NO:39)); PARP (KRKGDEVDGVDECAKKSKK (SEQ ID NO:40)), M9 peptide, NQSSNFGPMKGGNFGGRSSGPYGGGGQYFAKPRNQGGY (SEQ ID NO:41), and derivatives thereof.

15-18. (canceled)

19. The construct of claim 1, wherein:
said guide RNA comprises one or more bridged nucleic acids; or
said guide RNA comprises one or more bridged nucleic acids wherein said bridged nucleic acid comprises one or more N-methyl substituted BNAs (2′,4′-BNA^{NC}[N-Me]); or
said guide RNA comprises one or more locked nucleic acids (LNAs).

20-21. (canceled)

22. The construct of claim 1, wherein:
said targeting moiety binds to an internalizing receptor; or
said targeting moiety comprises an antibody binds to a receptor selected from the group consisting of CD45, CD3, erbB2, Her2, CD22, CD74, CD19, CD20, CD33, CD40, MUC1, IL-15R, HLA-DR, EGP-1, EGP-2, G250, prostate specific membrane antigen (PSMA), prostate specific antigen (PSA), prostatic acid phosphatase (PAP), and placental alkaline phosphatase; or
said targeting moiety comprises an antibody selected form the group consisting of OKT3, m291, foralumab, and CA-3.

23-26. (canceled)

27. The construct of claim 1, wherein said targeting moiety comprises an antibody.

28-33. (canceled)

34. The construct of claim 27, wherein:
said antibody is a full-length immunoglobulin; or
said antibody is selected from the group consisting of Fv, Fab, (Fab′)₂, (Fab′)₃, IgGΔCH2, a unibody, and a minibody; or
said antibody is, wherein said antibody is a single chain antibody; or
said antibody is an scFv; and/or
said antibody is a human antibody.

35-38. (canceled)

39. The construct of claim 1, wherein said targeting moiety is attached to said Cas endonuclease by a non-covalent interaction.

40. The construct of claim 39, wherein:
said non-covalent interaction comprises a biotin/avidin interaction; or
said non-covalent interaction comprises an interaction between an antibody-binding peptide and said targeting moiety; or
said non-covalent interaction comprises an interaction between said targeting moiety and a protein selected from the group consisting of Protein A, Protein G, Protein L, Protein Z, Protein LG, Protein LA, and Protein AG; or
said non-covalent interaction comprises an interaction between said targeting moiety and a moiety selected from the group consisting of PAM, D-PAM, D-PAM-θ, TWKTSRISIF (SEQ ID NO:4), FGRLVSSIRY (SEQ ID NO:5, Fc-III, EPIHRSTLTALL, HWRGWV (SEQ ID NO:7), HYFKFD (SEQ ID NO:8), HFRRHL (SEQ ID NO:9), NKFRGKYK (SEQ ID NO:10), NARKFYKG (SEQ ID NO:11), KHRFNKD (SEQ ID NO:12); or
said non-covalent interaction comprises an interaction between said targeting moiety and FcB6.1 peptide.

41-44. (canceled)

45. The construct of claim 1, wherein said antibody-binding peptide or said binding moiety is chemically conjugated to said Cas endonuclease via a cleavable linker.

46. The construct of claim 45, wherein:
said targeting moiety is chemically conjugated to said Cas endonuclease via a non-cleavable linker; or
said targeting moiety is chemically conjugated to said Cas endonuclease via a cleavable linker; or
said targeting moiety is chemically conjugated to said Cas endonuclease via a cleavable linker comprising a disulfide linker or an acid-labile linker; or
said targeting moiety is chemically conjugated to said Cas endonuclease via an acid label linker comprising a moiety selected from the group consisting of a hydrazone, an acetal, a cis-aconitate-like amide, a silyl ether.

47-54. (canceled)

55. The construct of claim 1, wherein said targeting moiety is chemically conjugated to said Cas endonuclease via a non-amino acid, non-peptide linker shown in Table 2.

56. The construct of claim 1, wherein said targeting moiety comprises a polypeptide and said targeting moiety and Cas endonuclease comprise a fusion protein.

57. The construct of claim 56, wherein:
said fusion protein comprises said targeting moiety directly attached to said Cas endonuclease; or
said fusion protein comprises said targeting moiety attached to said Cas endonuclease by an amino acid; or
said fusion protein comprises said targeting moiety attached to said Cas endonuclease by a peptide linker.

58-59. (canceled)

60. The construct of claim 57, wherein said fusion protein comprises said targeting moiety attached to said Cas endonuclease by a peptide linker wherein:
said peptide linker comprises an amino acid sequence cleavable by a protease; and/or
said peptide linker comprises an amino acid sequence cleavable by a cathepsin; and/or
said peptide linker comprises a dipeptide valine-citrulline (Val-Cit), or Phe-Lys.

61-62. (canceled)

63. The construct of claim 56, wherein said fusion protein comprises said targeting moiety attached to said Cas endonuclease by an amino acid or peptide linker shown in Table 2.

64. A pharmaceutical formulation, said formulation comprising a construct of claim 1, and a pharmaceutically acceptable carrier.

65-66. (canceled)

67. A method of performing gene editing on a cell, said method comprising contacting said cell with a construct of claim 1, wherein said guide RNA guides the Cas endonuclease to a specific location in the genome of said cell.

68-84. (canceled)
